SEQUENCE LISTING (38 sequences)

SEQ ID NO: 1 - DNA, 36 bp, Artificial Sequence
  primer
gaatgctacttttggacattaggagtttattttgag

SEQ ID NO: 2 - DNA, 32 bp, Artificial Sequence
  primer
ctcgcgtcatgctcgctaagaccatatcaatg

SEQ ID NO: 3 - DNA, 44 bp, Artificial Sequence
  primer
gaaagaatgaaagaaatagtaagaaattataatgtcgagtcaac

SEQ ID NO: 4 - DNA, 34 bp, Artificial Sequence
  primer
cctaagcaatgcactagcaacttccacatattac

SEQ ID NO: 5 - DNA, 25 bp, Artificial Sequence
  primer
ccacatattacttgctcgcgacaac

SEQ ID NO: 6 - DNA, 38 bp, Artificial Sequence
  primer
gcactagcaacttccacatattacttgctcgcgacaac

SEQ ID NO: 7 - DNA, 41 bp, Artificial Sequence
  primer
gaatccaaaaattcttgaagctaatgtaattatatgtcgag

SEQ ID NO: 8 - DNA, 34 bp, Artificial Sequence
  primer
cttgaagctagtgtaactatatgtcgagttatcg

SEQ ID NO: 9 - DNA, 39 bp, Artificial Sequence
  primer
cttgaagctagtgtaattctgtgtcgagttatcgatgac

SEQ ID NO: 10 - DNA, 41 bp, Artificial Sequence
  primer
aattcttgaagctaatgtaactatatgtcgagttatcgatg

SEQ ID NO: 11 - DNA, 43 bp, Artificial Sequence
  primer
aattcttgaagctaatgtaattctgtgtcgagttatcgatgac

SEQ ID NO: 12 - DNA, 43 bp, Artificial Sequence
  primer
aattcttgaagctaatgtaactctgtgtcgagttatcgatgac

SEQ ID NO: 13 - DNA, 39 bp, Artificial Sequence
  primer
cttgaagctagtgtaactctgtgtcgagttatcgatgac

SEQ ID NO: 14 - DNA, 39 bp, Artificial Sequence
  primer
ctcaatcttgctcgtattattgaggttacatatatacac

SEQ ID NO: 15 - DNA, 45 bp, Artificial Sequence
  primer
atgctttaaggctctgggccgacaagtttgtacaaaaaagcaggc

SEQ ID NO: 16 - DNA, 45 bp, Artificial Sequence
  primer
gtcacgcatatgattcggcggacaagtttgtacaaaaaagcaggc

SEQ ID NO: 17 - DNA, 45 bp, Artificial Sequence
  primer
cctggcttgctcggatagaacacaagtttgtacaaaaaagcaggc

SEQ ID NO: 18 - DNA, 45 bp, Artificial Sequence
  primer
ctacagagaatgccgcggtccacaagtttgtacaaaaaagcaggc

SEQ ID NO: 19 - DNA, 45 bp, Artificial Sequence
  primer
ggtcgtcgaccccagcgtgagacaagtttgtacaaaaaagcaggc

SEQ ID NO: 20 - DNA, 45 bp, Artificial Sequence
  primer
tcgcaattcacggcttgacccacaagtttgtacaaaaaagcaggc

SEQ ID NO: 21 - DNA, 45 bp, Artificial Sequence
  primer
aaatgcaggtagcagagctgtaccactttgtacaagaaagctggg

SEQ ID NO: 22 - DNA, 45 bp, Artificial Sequence
  primer
ttaggacgggactgctgtagcaccactttgtacaagaaagctggg

SEQ ID NO: 23 - DNA, 45 bp, Artificial Sequence
  primer
acattctgacgtgaaacgcgcaccactttgtacaagaaagctggg

SEQ ID NO: 24 - DNA, 45 bp, Artificial Sequence
  primer
ggacacacttagccttccaggaccactttgtacaagaaagctggg

SEQ ID NO: 25 - DNA, 45 bp, Artificial Sequence
  primer
ggcctggagtaggatctttgcaccactttgtacaagaaagctggg

SEQ ID NO: 26 - DNA, 45 bp, Artificial Sequence
  primer
cccgtcccacttcgtgaccgcaccactttgtacaagaaagctggg

SEQ ID NO: 27 - DNA, 21 bp, Artificial Sequence
  primer
atgctttaaggctctgggccg

SEQ ID NO: 28 - DNA, 21 bp, Artificial Sequence
  primer
gtcacgcatatgattcggcgg

SEQ ID NO: 29 - DNA, 21 bp, Artificial Sequence
  primer
cctggcttgctcggatagaac

SEQ ID NO: 30 - DNA, 21 bp, Artificial Sequence
  primer
ctacagagaatgccgcggtcc

SEQ ID NO: 31 - DNA, 21 bp, Artificial Sequence
  primer
ggtcgtcgaccccagcgtgag

SEQ ID NO: 32 - DNA, 21 bp, Artificial Sequence
  primer
tcgcaattcacggcttgaccc

SEQ ID NO: 33 - DNA, 21 bp, Artificial Sequence
  primer
aaatgcaggtagcagagctgt

SEQ ID NO: 34 - DNA, 21 bp, Artificial Sequence
  primer
ttaggacgggactgctgtagc

SEQ ID NO: 35 - DNA, 21 bp, Artificial Sequence
  primer
acattctgacgtgaaacgcgc

SEQ ID NO: 36 - DNA, 21 bp, Artificial Sequence
  primer
ggacacacttagccttccagg

SEQ ID NO: 37 - DNA, 21 bp, Artificial Sequence
  primer
ggcctggagtaggatctttgc

SEQ ID NO: 38 - DNA, 21 bp, Artificial Sequence
  primer
cccgtcccacttcgtgaccgc

(19) United States
(12) Patent Application Publication
(10) Pub. No.: US 2005/0233336 A1
(43) Pub. Date: Oct. 20, 2005

(54) Compositions and methods for producing libraries with controlled compositions and screening probabilities

(76) Inventors: Paul E. O'Maille, San Diego, CA (US); Joseph P. Noel, San Diego, CA (US)

Correspondence Address: KLARQUIST SPARKMAN, LLP, 121 S.W. SALMON STREET, SUITE 1600, PORTLAND, OR, 97204, US

(21) Appl. No.: 10/827,914

(22) Filed: Apr. 19, 2004

(51) Int. Cl. C12Q 1/68; C12P 19/34

(52) U.S. Cl. 435/6; 435/91.2

(57) ABSTRACT

The invention provides a method for the combinatorial mutagenesis of a parental nucleic acid. The method consists of: (a) extending by enzymatic polymerization a first mutagenic primer annealed to a parental nucleic acid to produce an extension product; (b) treating said extension product with a cleaving reagent selective for a nucleotide sequence present in the parental nucleic acid but absent in the first product; (c) extending by enzymatic polymerization a first PAP annealed to a noncontiguous region of said mutagenic primer to produce a first product having a first mutagenized portion comprising one or more altered nucleotides, the first PAP containing a unique sequence tag associating mutations within the first mutagenic primer with the first PAP; (d) annealing the first product to the parental nucleic acid, and (e) extending by enzymatic polymerization the annealed first product to produce a first modified parental nucleic acid containing a first mutagenized portion. The first product can additionally be amplified. The method also provides the additional step: (f) amplifying the first modified parental nucleic acid containing a first mutagenized portion by polymerase extension of an annealed first SAP to the unique sequence tag contained in the first PAP and an annealed second PAP to the first modified parental nucleic acid, the first and second PAPs corresponding to flanking regions of the parental nucleic acid. The method additionally provides the steps of: (g) repeating steps (a) through (c) one or more times with a second mutagenic primer and a third PAP to noncontiguous regions of the parental nucleic acid to a second product having a second mutagenized portion, the third PAP containing a unique sequence tag associating mutations within the second mutagenic primer with the second PAP, and (h) repeating steps (d) through (e) or steps (d) through (f) one or more times by annealing the second product produced in step (g) to the parental nucleic acid or the first modified parental nucleic acid produced in step (e) or (f) to generate a second modified parental nucleic acid containing a first mutagenized portion and at least one second mutagenized portion. Steps (g) and (h) can be repeated one or more times with tertiary mutagenic primers.

This invention was made with government support under grant numbers GM54029 or GM069056-01 awarded by the National Institutes of Health. The United States Government has certain rights in this invention.

### BACKGROUND OF THE INVENTION

This invention relates generally to nucleic acid synthesis and, more specifically to the design and construction of diverse libraries of variant nucleic acids.

The synthetic construction of diverse populations of polypeptides has been one focus in the research and discovery of novel biotherapeutics. The polypeptide populations are produced by expressing diverse populations of encoding nucleic acids and then screened for gene products exhibiting the preferred activities. One approach has encompassed creating random populations of polypeptide of sufficient diversity to screen for the polypeptide having the sought characteristics. Other approaches include searching the genome of diverse organisms for related polypeptides having untapped sequence variability that may exhibit useful functions or generating variants of polypeptides and screen for the desired changes in activity. For example, optimization of a polypeptide's activity has been attempted by screening of natural sources, or by use of mutagenesis. In particular, site-directed mutagenesis results in substitution, deletion or insertion of specific amino acid residues chosen either on the basis of their type or on the basis of their location in the secondary or tertiary structure of the mature enzyme.

One method for the recombination between two or more nucleotide sequences of interest involves shuffling homologous DNA sequences by using in vitro polymerase chain reaction (PCR) methods. Nucleic acid recombination products containing shuffled nucleotide sequences are selected from a DNA library based on the improved function of the expressed proteins. A disadvantage inherent to this method is its dependence on the use of homologous gene sequences and the production of random fragments by cleavage of the template double-stranded polynucleotide. In particular, recombination between nucleotide sequences requires sufficient sequence homology to enable hybridization of the different sequences, the inherent disadvantage is that the diversity generated is relatively limited. This homology limitation also inherently restricts the application of site-directed mutagenesis because of the requirement for sequence similarity between sequences that are to be recombined.

Other methods for creating diverse populations require intricate synthesis procedures or separation steps to ensure a reduced background levels of undesirable nucleic acids from the mixture. These procedures and steps can be labor intensive or require automation when a large number of product sequences are desirable. While methods exist for making nucleic acid library populations encoding shuffled polypeptides of similar sequence or mutagenized species, there is yet no efficient method that allows incorporation of altered nucleotide sequences into a parental sequence without substantial manipulation or sequence homology.

The goal of library synthesis techniques is the creation of sequence space. Every position in a polypeptide chain is one of 20 possible amino acids, and so for a protein with 100 amino acids, there are 20¹⁰⁰ possible sequences. It is extremely difficult, if not impossible, to create libraries of this size at least because there is neither sufficient time nor a sufficient amount of carbon source available for generating these molecular populations. Instead, discrete combinations of mutations are made. As mixtures of mutant oligonucleotides become more complex however (as the variety increases), the sampling requirements giving a fixed probability of screening the complexity (picking 1 of each unique representative out of a complex mixture) increases exponentially

Thus, there exists a need for a method of making diverse populations of altered nucleic acids that is efficient and accurate. The present invention satisfies this need and provides related advantages as well.

### SUMMARY OF THE INVENTION

The invention provides a method for the combinatorial mutagenesis of a parental nucleic acid. The method consists of: (a) extending by enzymatic polymerization a first mutagenic primer annealed to a parental nucleic acid to produce an extension product; (b) treating said extension product with a cleaving reagent selective for a nucleotide sequence present in the parental nucleic acid but absent in the first product; (c) extending by enzymatic polymerization a first PAP annealed to a noncontiguous region of said mutagenic primer to produce a first product having a first mutagenized portion comprising one or more altered nucleotides, the first PAP containing a unique sequence tag associating mutations within the first mutagenic primer with the first PAP; (d) annealing the first product to the parental nucleic acid, and (e) extending by enzymatic polymerization the annealed first product to produce a first modified parental nucleic acid containing a first mutagenized portion. The first product can additionally be amplified. The method also provides the additional step: (f) amplifying the first modified parental nucleic acid containing a first mutagenized portion by polymerase extension of an annealed first SAP to the unique sequence tag contained in the first PAP and an annealed second PAP to the first modified parental nucleic acid, the first and second PAPs corresponding to flanking regions of the parental nucleic acid. The method additionally provides the steps of: (g) repeating steps (a) through (c) one or more times with a second mutagenic primer and a third PAP to noncontiguous regions of the parental nucleic acid to a second product having a second mutagenized portion, the third PAP containing a unique sequence tag associating mutations within the second mutagenic primer with the second PAP, and (h) repeating steps (d) through (e) or steps (d) through (f) one or more times by annealing the second product produced in step (g) to the parental nucleic acid or the first modified parental nucleic acid produced in step (e) or (f) to generate a second modified parental nucleic acid containing a first mutagenized portion and at least one second mutagenized portion. Steps (g) and (h) can be repeated one or more times with tertiary mutagenic primers.

### BRIEF DESCRIPTION OF THE DRAWINGS

FIG. 1 shows a schematic overview of the SCOPE library synthesis process.

FIG. 2 shows a schematic overview of SCOPE-based combinatorial mutagenesis. FIG. 2A illustrates a source of background wild-type sequence accumulation. FIG. 2B illustrates an alternative fragment amplification strategy for the suppression of wild-type sequence.

FIG. 3 shows a schematic representation of amino acid mutations introduced into tobacco 5-epi-aristolochene synthase (TEAS) by SCOPE-based combinatorial mutagenesis of an encoding parental nucleic acid.

FIG. 4 shows recombination units and tagging system that can be used to describe recombination products created by SCOPE-based combinatorial mutagenesis. Recombined mutant positions and their associated unique sequence tags are depicted in the chart in the upper panel. The lower panel depicts incorporation of the recombined mutant positions through the SCOPE combinatorial mutagenesis process and illustrates the structural organizational of the recombination product designated by the recombination unit and tagging nomenclature.

FIG. 5 shows the sampling probability as a function of over-sampling for a population of variant molecules produced by combinatorial mutagenesis. The probability that a sample contains one copy of each unique clone for a given complexity (n) is calculated using equation (1). Probability is calculated for a range of sample sizes (k) that are in multiples of a fixed library complexity (n) and the results are fit to a sigmoidal curve.

FIG. 6 shows the product specificity of two closely related terpene cyclases, Hyoscyamus muticus Premnaspirodiene Synthase (HPS) and Tobacco 5-Epi-Aristolochene Synthase (TEAS).

### DETAILED DESCRIPTION OF THE INVENTION

This invention is directed to combinatorial mutant library design and construction that allow both homology-independent recombination and combinatorial mutagenesis. The combinatorial mutagenesis methods of the invention enable the synthesis of mutant libraries with controlled compositions and a predetermined probability of screening the diversity. An advantage of the methods of the invention is that they provide an effective means for either or both the creation of global or local sequence variability or diversity. Additionally, the methods of the invention offer a combinatorial approach to synthesize mutant libraries of selected mutations irrespective of the distance between the individual muttions.

In one embodiment, the invention is directed to the systematic incorporation of mutations into a parental nucleic acid using a mutagenic primer and polymerase chain reaction (PCR). The second primer includes a sequence tag that uniquely identifies the mutagenic sequence. Synthesis or amplification using the mutagenic primer and tagged primer produces a parental nucleic acid product containing the mutations and the unique tag. Iterative cycles with different mutagenic and tagged primers result in a population of modified parental nucleic acids harboring a diverse number of mutations. Each mutant species can be identified by deconvolution of the population and correlation of the unique tags to their associated mutagenic sequences. In another embodiment, the invention is directed to the systematic incorporation of a plurality or a diverse plurality of mutations into one or more parental nucleic acid sequences. The unique sequence tags employed in association with a specified mutagenic primer sequence allows for such multiplexing of the combinatorial mutagenesis method of the invention.

As used herein, the term “combinatorial mutagenesis” is intended to mean the synthesis of a number of different but related nucleic acids in order to produce variants of a parental nucleic acid. The related nucleic acids similarly encode a number of different but related polypeptides. The variant nucleic acids or encoded polypeptides can be screened to identify molecular species optimally suited for a specific function.

As used herein, the term “parental” when used in reference to a nucleic acid is intended to mean a progenitor molecule of a variant nucleic acid or encoded polypeptide of the invention. A parental nucleic acid includes the starting nucleic acid species that is mutagenized by the methods of the invention. A parental nucleic acid also includes intermediate species that have undergone one or more rounds of combinatorial mutagenesis, but which are employed as a starting molecule or template in subsequent iterations of fragment amplification, recombination, extension or amplification. Generally, a parental molecule will correspond to a wild-type gene or genomic sequence but can include, for example, chimeric and other forms of a reference sequence that is a target for incorporation of mutations. The term also can be used in reference to multiple species or different variants of a reference sequence. For example, a multiplex analysis can contain, for example, from one to many starting targeted reference sequence for which a diverse population of variants is desired to be produced. The starting targeted reference sequences can be similar or divergent in sequence similarly. Similarly, the plurality of first, second or tertiary products or first, second or tertiary modified parental nucleic acids also are included within the meaning of the term when used in reference to a target template employed in subsequent rounds of combinatorial mutagenesis.

As used herein, the term “modified parental product” is intended to mean that the nucleotide sequence of a parental nucleic acid has been changed. Nucleotide changes are referred to herein as mutations, alterations, variants or equivalent grammatical forms thereof. A modified parental product therefore has a mutant, altered or variant nucleotide sequence compared to the sequence of its parental nucleic acid. A modified parental product includes, for example, first, second and tertiary modified parental products.

As used herein, the term “primer” is intended to mean a polynucleotide that is complementary to a portion of a target nucleic acid and can anneal and promote template-directed polymerase extension of a nucleic acid product. The target nucleic acid can be, for example, a parental nucleic acid or a first, second or tertiary modified nucleic acid.

As used herein, the term “mutagenic primer” is intended to mean a nucleic acid complementary to a portion of a parental sequence and containing at least one nucleotide different from the complement of the parental sequence portion. When annealed to a parental nucleic acid, mutagenic primers can direct polymerase extension and incorporation of the altered nucleotide into the extension product. Mutagenic primers therefore direct the mutagenesis of a parental nucleic acid and correspond to a mutagenic sequence. The nucleic acid primer also can be complementary to a portion of first, second or tertiary modified product or other nucleic acid derived from a recombination or amplification step and employed as a template for incorporation of mutations. Enzymatic methods other than polymerase extension which allow incorporation of altered sequences of an oligonucleotide into a template also can be employed with the mutagenic primers of the invention.

As used herein, the term “primary amplification primer” or “PAP” is intended to mean a nucleic acid primer that is complementary to a termini region or to a flanking region of a sequence to be mutagenized. The terminal or flanking region can be located in relatively close proximity or distally to the targeted mutation region. PAPs can uniquely identify or be modified to uniquely identify a mutagenic primer, or its mutagenized product, with the corresponding terminal or flanking region targeted sequence. For example, inclusion of additional sequence or structural information in the PAP can be used to uniquely associate a mutagenic primer with the terminal or flanking region of the mutagenic target. In the specific instance where a parental nucleic acid targeted for combinatorial mutagenesis is a gene encoding a polypeptide product, a PAP generally will correspond to the either the 5′ or 3′ external region of the gene or external sequences flanking these regions. However, internal regions corresponding to the terminal portion of a smaller target region also can correspond to PAP sequence used in the methods of the invention.

As used herein, the term “secondary amplification primer” or “SAP” is intended to mean a nucleic acid primer that is complementary to an identifying sequence tag contained within a PAP. Therefore, a SAP can anneal to a PAP nucleotide sequence and promote template-directed polymerase extension of a PAP containing nucleic acid sequence. The PAP containing sequence includes, for example, a parental nucleic acid or a first, second or tertiary modified nucleic acid.

As used herein, the term “unique” when used in reference to a sequence tag associated with a PAP is intended to mean that the tag has a sequence distinguishable from other sequences in the same mixture. Therefore, a unique sequence tag can be detectable as a separate component or entity within a mixture by a recognizable difference. A unique sequence tag is useful, for example, to associate an mutagenic sequence with a parental sequence primed by a PAP.

As used herein, the term “noncontiguous” when used in reference to regions of a nucleic acid sequence is intended to mean a non-adjoining region to the reference nucleic acid region. Therefore, a noncontiguous region of a parental nucleic acid is not immediately preceding or following a parental nucleic acid region of reference. The distance between noncontiguous regions will be sufficient to allow enzymatic polymerization of an annealed primer. Accordingly, noncontiguous regions can be separated by short distances, such as from one or a few nucleotides, and can be separated by large distances, such as by tens, hundreds or thousands of nucleotides.

The invention provides a method for the combinatorial mutagenesis of a parental nucleic acid, comprising: (a) extending by enzymatic polymerization a first mutagenic primer and a first PAP annealed to noncontiguous regions of a parental nucleic acid to produce a first product having a first mutagenized portion comprising one or more altered nucleotides, the first PAP containing a unique sequence tag associating mutations within the first mutagenic primer with the first PAP; (b) treating the first extension product or the first product with a cleaving reagent selective for a nucleotide sequence present in the parental nucleic acid but absent in the first product; (c) annealing the first product to the parental nucleic acid, and (d) extending by enzymatic polymerization the annealed first product to produce a first modified parental nucleic acid containing a first mutagenized portion. The first extension product or first product treated with the cleaving reagent also can be amplified.

The method for combinatorial mutagenesis can further include step (e) amplifying the first modified parental nucleic acid containing a first mutagenized portion by polymerase extension of an annealed first SAP to the unique sequence tag contained in the first PAP and an annealed second PAP to the first modified parental nucleic acid, the first and second PAPs corresponding to opposite termini of the parental nucleic acid. Step (a) can be repeated one or more times with a second mutagenic primer and a third PAP to noncontiguous regions of the parental nucleic acid to produce a second product having a second mutagenized portion, the third PAP containing a unique sequence tag associating mutations within the second mutagenic primer with the second PAP, and can include the additional step: (g) repeating steps (b) through (d) or steps (b) through (e) one or more times by annealing the second product produced in step (f) to the parental nucleic acid or the first modified parental nucleic acid produced in step (d) or (e) to generate a second modified parental nucleic acid containing a first mutagenized portion and at least one second mutagenized portion. Further, the method of combinatorial mutagenesis also can include step (h) repeating steps (f) and (g) at least once with one or more tertiary mutagenic primers and tertiary PAPs to generate a tertiary modified parental nucleic acid containing first, second and tertiary mutagenized portions.

Structure-based combinatorial protein engineering, referred to herein as SCOPE, is a process for the synthesis of populations of nucleic acids. SCOPE is useful as a tool for exploring the relationship between structure and function of a polypeptide. The combinatorial mutagenesis methods described herein allow for an exhaustive dissection, identification and assignment of function to primary, secondary or tertiary structures of polypeptides or encoding nucleic acids. The combinatorial mutagenesis methods can employ the SCOPE process.

Comparative analysis of polypeptide structure can be used to assess relationships between molecular structure and functional activity. SCOPE facilitates construction of nucleic acid populations that encode rationally engineered polypeptide variants that can be used in such comparative analyses. Structural models generated from experimental data such as crystallographic methods, NMR methods and homology modeling can be used to design nucleic acid primers that code for crossovers between genes encoding structurally related proteins. A series of polymerase chain reactions (PCR) can be used to produce selective amplification of crossover products. The products incorporate spatial information encoded in the nucleic acid primer into a full-length encoding nucleic acid or gene and the resultant hybrid polypeptide. Iteration of the process enables the synthesis of many possible combinations of desired crossovers, producing a hierarchical collection of chimeras in analogy to a Mendelian population.

The principles of SCOPE and the combinatorial mutagenesis methods described herein are generally applicable and easily adapted to a range of practical and research objectives. SCOPE provides a homology-independent in vitro recombination approach for generating multiple-crossover gene libraries from distantly related polypeptides (O'Maille et al., J. Mol. Biol. 321:677 (2002)).

SCOPE-based combinatorial mutagenesis enables the facile combinatorial synthesis of diverse populations of variant nucleic acid or gene libraries. The combinatorial methods described herein provide a robust and efficient method for the determination of structure and functional relationships as well as the identification of polypeptide variants or the creation and identification of new functions in a multidimensional polypeptide or nucleic acid sequence space. The structural or functional information obtained from the combinatorial mutagenesis methods of the invention as well as the chimeric or variant polypeptides and encoding nucleic acids are useful in a wide range of therapeutic, diagnostic or research applications.

The combinatorial mutagenesis methods of the invention are equally applicable to, for example, all forms of nucleic acids and encoded polypeptides. For example, the methods of the invention can be employed to create a diverse population of variant encoding nucleic acids for the association of a polypeptide secondary or tertiary structure to its primary amino acid or encoding nucleic acid sequence. Combinatorial mutagenesis is similarly applicable to, for example, nucleic acid regulatory regions, introns or intervening regions within a genomic nucleic acid fragment. In the former example, the structure and/or functional attributes, identification of new variants or creation of new functions are assessed at the polypeptide level, including all molecular interactions integrated with the target structure or function. In the latter example, these attributes, variants or new functions are instead assessed at the nucleic acid level and also include the various integrated molecular interactions. Therefore, the combinatorial mutagenesis methods of the invention are equally applicable to nucleic acids corresponding to coding, non-coding or genomic regions.

The invention will be described with reference to combinatorial mutagenesis of coding nucleic acid sequences and their encoded polypeptide structures and functions. However, given the teachings and guidance provided herein, those skilled in the art will understand that the methods of the invention can be readily applied to non-coding nucleic acid sequences as well as to other types of macromolecules composed of monomer building blocks similar to the nucleotide and amino acid building blocks of nucleic acids and polypeptides.

As stated previously, construction of gene libraries by SCOPE involves a series of parallel or sequential PCR reactions. Other recombination techniques use multiple primers or random fragments in a single step. Separation of gene synthesis into discrete steps allows the user to control recombination through pairing gene fragments and genes that give rise to designed and anticipated combinations of crossovers. As a consequence, libraries are constructed as a series of less complex mixtures, which reduces numerical complexity and the cost and extent of sampling required during screening, including gene sequencing and functional assays. Crossover locations and the frequency of genetically encoded crossovers are established by experimental design and are devoid of homology constraints between genes or the linear distance between multiple mutations. As described herein, genes correspond to parental nucleic acids. Gene fragments correspond to first, second and tertiary products of the combinatorial mutagenesis methods of the invention.

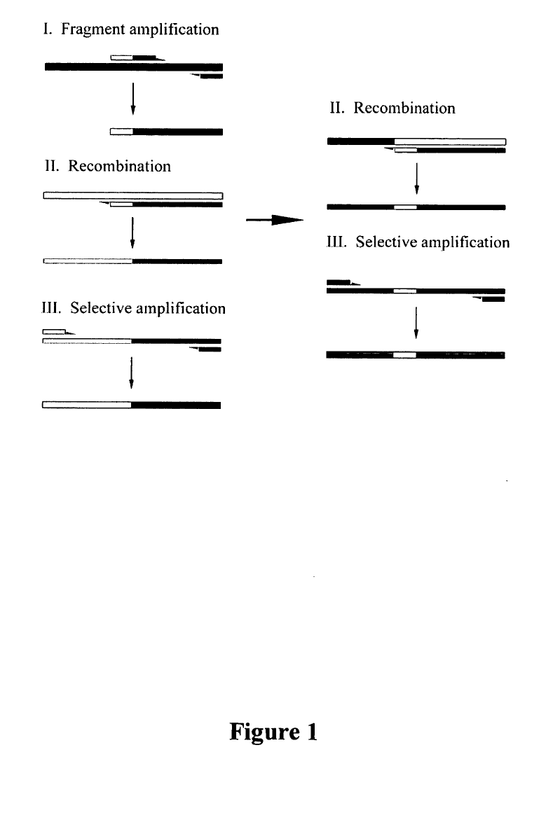

Recombination based on SCOPE is illustrated in FIG. 1. Briefly, in step I, PCR amplification employing an internal and external primer pair and the appropriate template DNA is used to produce chimeric gene fragments. Internal primers can be designed on the basis of one or more encoded three-dimensional structures viewed with reference to the variable sequence space of protein homologues and code for crossovers in the protein-coding region of genes. External primers can correspond to the 5′ and 3′ termini of a given gene, similar to primer pairs used in PCR amplification of a coding region sequence. Amplification template can consists of, for example, an amplification target harbored in a plasmid or a PCR product that contains the gene of interest or any other form of nucleic acid alone or contained in a vehicle useful for recombinant manipulation.

In step II, in vitro recombination occurs between a gene fragment and a new template such as a parental nucleic acid or a parental nucleic acid corresponding to a first, second or tertiary modified parental nucleic acid. For example, amplified gene fragments can serve as primers sets for new rounds of amplification of the target gene or parental nucleic acid. Such primers can be annealed and extended to produce single-stranded full-length chimeras corresponding to the two parent sequences for which recombination is to occur.

In step III, a new external primer set directs the selective amplification of the final recombination products or chimeras. This final primer set can be selected by virtue of a unique genetic identity encoded at the termini of the resultant chimeras. Repetition of steps II and III using predetermined pairs of gene fragments from step I and crossover products from step III, allows the production of genetically diverse, multiple crossover libraries of the parent sequences in high yield.

The SCOPE recombination process employs oligonucleotide primers designed to amplify selected segments of a parental nucleic acid target gene to yield recombination between two parents at a predetermined location. The relationship of oligonucleotide primers to specific amplification or recombination applications is exemplified below and illustrates the adaptation of SCOPE for the construction of multiple crossover libraries from distantly related proteins or for the construction combinatorial mutant libraries from functionally related or unrelated polypeptides.

Internal primers can be employed for the shuffling of exons or equivalent structural elements between gene homologues. The internal primers can have a chimeric structure, for example, consisting of nucleotide sequences corresponding to each of the two parental sequences and coding for a crossover region. In this regard, about one half of the primer can correspond to a first parental sequence beginning 5′ to the crossover junction and terminating at the crossover junction. The other half of the primer can correspond to a second parental sequence beginning at the crossover junction and ending 3′ to the junction. An example of such internal use is illustrated in step I of FIG. 1. Absence of prior knowledge of the optimal point of fusion in regions of low identity or the compatibility of equivalent structural elements of low sequence identity, linkage variability can be introduced into the internal primers to accomplish recombination between parental genes. Linkage variability entails designing a set of chimeric oligonucleotides corresponding to a given crossover region, which code for a series of insertions, deletions or both, around a fixed crossover point.

Following amplification, the corresponding collection of gene fragments can be used in subsequent recombination reactions such as that illustrated in step II of the SCOPE process or combinatorial mutagenesis methods described herein. Variable connections between equivalent structural elements provide design advantages that result in the efficient production of functional chimeras from distantly related DNA polymerases (O'Maille et al., supra, 2002).

Combinatorial mutagenesis employing SCOPE can utilize mutagenic oligonucleotides that generate, for example, variations at one or more nucleotide or encoding amino acid positions. The incorporated variations can be, for example, specific changes at selected positions; random, degenerate or biased variations at one or more residues or random, degenerate or biased sets of variant residues. Such variations can include, for example, changes of single or multiple nucleotide or encoding amino acid residues as well as insertions, deletions or other modification formats well known to those skilled in the art that can be directed to a specific site or region within a parent gene. The variant residues introduced also can be contiguous within a linear primary sequence or non-contiguous across a primary sequence. Alternatively, bridging oligonucleotides, which code for stretches of native sequence between mutations, can be used to mediate recombination between parental genes and/or variant genes. Mutagenic and bridging oligonucleotides are employed in amplification reactions similarly to chimeric oligonucleotides. Amplification reactions can include linear amplification such as by polymerase extension or exponential amplification such as by PCR. The modifications described below additionally can be used to increase the efficiency of mutagenic and bridging oligonucleotide incorporation into the final product.

External primers can be employed, for example, in the final step of the cycle for the amplification of mutagenized genes. The use of external primers is illustrated in step III of FIG. 1. Amplification of the mutagenized gene can be accomplished using a primer set that flanks the region encompassing the mutagenized region. Additionally, the inclusion of restriction or recombination sites into the final primer set can be utilized for efficient cloning or other manipulations of the resultant collection of genes.

Primer design for selective amplification of a particular crossover product from a recombination reaction using SCOPE, for example, can depend on the desired intermediate crossover product or population of final chimeric products. For the chimeragenesis of distantly related polypeptides, the termini of each gene will generally be unique and can be utilized as primer binding sites for selective amplification of the desired intermediate or final chimeric crossover product or products. The amplification reaction can be designed to result in single, multiple or a diverse plurality of different crossover products from one or more recombination reactions.

Primer design for SCOPE-based combinatorial mutagenesis differs from SCOPE-based protein engineering, in part, because a purpose of combinatorial mutagenesis is to produce variants of the same or similar parental polypeptides. The variant or mutagenized sequence regions can be in one or different structural or functional domains of the encoded polypeptide. Whereas a purpose of SCOPE-based protein engineering is to produce recombination products between evolutionary related polypeptides in order to decipher the relationship between a particular structure and the function in confers on the polypeptide. In general, the initial parental molecules in combinatorial mutagenesis will consist of wild-type genes and encode wild-type gene products. In such instances, the parental molecules used in combinatorial mutagenesis will have, for example, the same or similar nucleic acid or encoded polypeptide sequence. Accordingly, the regions of parental molecules, such as sequences flanking a region of interest or the termini of the parental molecule, also will be indistinguishable among the variants produced and, absent further modifications, unable to be exploited for selective amplification by primer annealing in SCOPE-based engineering.

To impart sequence specificity onto terminal regions of parental molecules employed in SCOPE-based combinatorial mutagenesis, external primers can be designed, for example, with unique sequence tags. When used in conjunction with a classification system, the tagged external primers can be implemented to maintain a hierarchical organization and storage system for creating the mutagenized recombination products and diverse populations of variant chimeric products.

For example, primary amplification primers (PAPs) code for DNA sequences flanking a gene and additionally include a unique 5′ sequence tag. Use of PAPs in step I for mutagenized gene fragment synthesis links a unique sequence to a particular mutation. Following recombination, secondary amplification primers (SAPs), which correspond to the 5′ unique sequence encoded in a PAP, are employed in the final amplification (step III) to select for the desired recombination products, consisting of the parental nucleic acid sequence harboring the newly incorporated mutation or mutations.

In addition to tagged external primers and the hierarchical classification system described herein, additional procedural modifications can be implemented to increase incorporation efficiency of, for example, unique sequence tags, their linkage to mutations, the suppression of wild-type background genes or any combination of these attributes. Such modifications can include, for example, restriction or other enzymatic or chemical step that selectively destroys undesirable parental or intermediate templates in the reaction mixtures in order to enrich amplification of the designed variant population products.

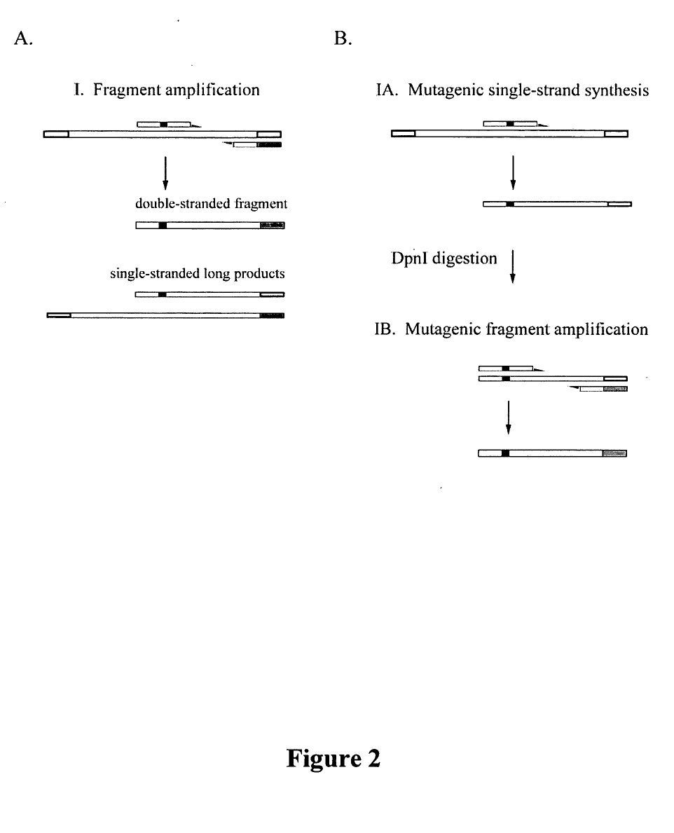

For example, during step I amplification, single-stranded DNA or “long” product is produced from extension of each primer on the plasmid template. As shown in FIG. 2A, when these single-stranded products are derived from PAPs they code for the wild-type gene. If such wild-type gene templates are carried over into other recombination or amplification steps of the process, they will give rise to a small but significant background population of wild-type genes. Separating step I into two reactions alleviates wild-type sequence contamination. For example, in FIG. 2B, step IA, internal primer and template are mixed and single-stranded DNA containing the mutation or population of mutations is synthesized. The product of step IA can be treated with a restriction enzyme such as Dpn I to digest the wild-type plasmid template, leaving only the nascent, single-stranded, mutagenic DNA. This restriction step eliminates the formation of long products that contribute to wild-type background. A portion of step IA product is then used in step IB, where it can serve as template for PCR or other amplification procedure with an internal primer and a PAP. Enzymatic digestion or other means of removing parental sequences from recombination or amplification reactions eliminates the need for physical or biochemical separation procedures in order to achieve the same or better results. Accordingly, the above modification enables the entire series of amplification reactions (steps I through III) to be conducted without purifying intermediates.

The basic steps outlined above for the combinatorial mutagenesis of a parental nucleic acid can be used, for example, to produce mutagenized nucleic acid populations containing directed nucleotide changes in single, double or multiple regions of a parental nucleic acid. Various permutations and combinations of these steps as described herein or known to those skilled in the art also can be implemented to augment the mutagenesis or modify the methods to obtain a desired outcome. Given the teachings herein, those skilled in the art will understand that a variety of recombinant manipulations or modifications can be incorporated into the methods described herein while still obtaining the mutagenized populations of the invention.

Combinatorial mutagenesis can be implemented in sequential or parallel synthesis formats. Additionally, multiplex synthesis of the mutagenized nucleic acid populations also can be readily performed by inclusion of multiple synthesis or amplification primers specific for different parental nucleic acids and each pair of primers having a unique association between a mutagenic primer and a unique sequence tag. Nucleic acid synthesis can be enzymatic polymerization in a template-directed manner from one or more primers annealed to a parental nucleic acid template. Depending on the need and desired outcome of the user, such enzymatic synthesis can be, for example, production of a duplicate nucleic acid strand, linear amplification directed from a primer annealed to one strand of a parental nucleic acid template or exponential amplification directed from primers annealed to opposite strands of a parental nucleic acid. The desired yield, amount of starting material and number of synthesis rounds are some factors well known to those skilled in the art which can be adjusted to generate a product population at a desired efficiency. Given the teachings and guidance provided herein as well as that known in the art adjustment of such parameters is well within the skill of one in the art.

Parental nucleic acids that can be employed in the combinatorial mutagenesis methods of the invention can include any nucleic acid molecule in which one or more nucleotide changes are desired. Such nucleic acids include, for example, genomic DNA, cDNA or RNA. Regions that can be mutagenized within such nucleic acids can include, for example, coding regions, non coding regions such as 5′ or 3′ untranslated regions, introns, regulatory sequences such as promoter or regulatory sequences, intervening sequences and the like. The nucleotide changes can be incorporated at a single region, a few regions or multiple regions. Such regions targeted for mutagenesis or mutagenic regions can be close together, dispersed, randomly dispersed or overlapping, for example. Accordingly, the methods of the invention are applicable to all forms of nucleic acids ranging from genomic sequences to synthetic oligonucleotides.

Combinatorial mutagenesis can be performed through iterative sequential, parallel or multiplex amplification steps where each step incorporates primer directed mutations into a parental nucleic acid to produce a nucleic acid product harboring the mutations. The nucleic acid product can be subsequently used as a primer for a further amplification step to recombine or join the mutagenic product with the remainder of the parental nucleic acid sequence. The recombined mutagenic product portion and parental sequence portion results in a modified parental nucleic acid containing the mutations. The modified parental nucleic acid can be, for example, screened directly for a desired activity or amplified and screened. Incorporation of further primer directed mutations can be achieved by further rounds of the above steps employing the modified parental nucleic acid as a parental nucleic acid for primer directed mutagenesis. Further, identifying incorporated mutations can be accomplished by using, for example, a unique sequence tag associated with a second primer used in the initial amplification step.

Primer directed mutagenesis can be accomplished, for example, by employing a pair of associated primers in a PCR reaction. One primer of the pair consists of a mutagenesis primer and is employed to direct the incorporation of one or more nucleotide changes at a predetermined region of a parental nucleic acid sequence. The second primer of the pair consists of a primary amplification primer (PAP), which is employed to prime the parental nucleic acid template at a noncontiguous region downstream from the mutagenic primer. It will be understood by those skilled in the art that the terms downstream and upstream when used in reference to nucleic acid primers for primer-template directed polymerase extension are relative terms and can correspond to either the 5′ or 3′ end because of the double-stranded anti-parallel nature of DNA.

A first round of combinatorial mutagenesis is initiated by synthesis of a first product having a first mutagenized portion corresponding to the mutagenic primer which directs nucleotide alterations of the parental nucleic acid. In many instances, regions to be altered will generally reside internally within the parental nucleic acid sequence. However, incorporation of mutations using a mutagenic primer can be performed either internally or at a parental nucleic acid terminus following the methods of the invention. Because the mutagenic primers will generally correspond to internal regions, following amplification, the first product generated also will generally correspond to a fragment of the parental nucleic acid.

The PAP employed as the second primer of the pair will correspond to a noncontiguous region of the parental nucleic acid sequence. The noncontiguous sequence can be, for example, a terminal region or an internal region so long as it resides at a noncontiguous location compared to the region to be altered by the mutagenic primer. Generally, the noncontiguous region primed by a PAP will correspond to a terminal region of the parental nucleic acid. Each PAP of a primer pair can contain, for example, a unique sequence tag. The sequence tag is chose so that it is of sufficient complexity to ensure uniqueness compared to the parental nucleic acid and compared to the mutagenic primer as well as other primers and tags employed in the same or subsequent rounds of combinatorial mutagenesis. Additionally, the unique sequence tag is designed and used in combination with a specific mutagenic primer such that there is a one-to-one correspondence, for example, between the mutagenic sequence and the unique sequence tag with a primer pair used in first product synthesis.

Accordingly, a unique sequence tag will correspond to, for example, an exogenous, synthetic or non-homologous sequence that lacks sequence similarity or identity to other sequences with the combinatorial mutagenesis reaction mixture. Similarly, a unique sequence tag also will lack sequence similarity or identity to other sequences present in reaction mixtures in subsequent iterations of the combinatorial mutagenesis method steps of the invention. Such other sequences include, for example, parental nucleic acid sequences, PAP sequences, SAP sequences other than the cognate SAP designed to be complementary to the unique sequence tag, or mutagenic primer sequences.

The number of unique sequence tags required for a particular combinatorial mutagenesis procedure will be determined, for example, based on the number of initial parental nucleic acids and the number of mutagenic regions used to incorporate a designed set of mutations. In this regard, the combinatorial mutagenesis methods of the invention will use a one-to-one correspondence between each mutagenic primer and corresponding PAP. In the simple instance where there is a single parental nucleic acid and two mutagenic regions, each with corresponding mutagenic primers, the number of unique sequence tags utilized in first product synthesis will be two. One unique sequence tag will correspond to, and uniquely identify, each of the mutagenic primers. In more complex instances where, for example, there is a single parental nucleic acid and many mutagenic regions, each also having a corresponding mutagenic primer, the number of unique sequence tags utilized in first product synthesis will be equal to the number of mutagenic regions. In very complex instances where, for example, there are multiple parental nucleic acids and many mutagenic regions within each parental nucleic acid and having a corresponding number of mutagenic primers, the number of unique sequence tags will be equal to the sum of the total number of mutagenic regions for all parental nucleic acids. Similarly, as additional mutations are incorporated in iterative rounds using, for example, first, second or tertiary modified parental nucleic acids as a parental nucleic acid for combinatorial mutagenesis, the unique sequence tags also should exhibit the criteria outlined above. Namely, the sequences should, for example, uniquely identify the mutagenic sequence associated with each new PAP within the additional primer pairs. Additionally, the same PAP can be used for different mutational regions, so provided that the corresponding first, second or tertiary modified parental nucleic acids are employed in separate reactions, only a limited number of unique sequence tags are needed (less than the number of mutations).

Unique sequence tags can consist of essentially any sequence or combination of sequences so long as the nucleotide sequence of each tag is unique within the reaction mixture or can be made to uniquely identify the parental nucleic acid template. For example, the length of unique sequence tags and complexity can depend, for example, on the complexity of the reaction mixture, size of the parental nucleic acid or number of parental nucleic acid species present in the synthesis reaction mixture. Sequence complexity, sequence homology and uniqueness compared to other nucleotide sequences are well known to those skilled in the art. For example, those skilled in the art can determine the extent of sequence similarity by aligning the sequences with an algorithm such as BLAST (Altschul et al., J. Mol. Biol. 215:403-410 (1990)), WU-BLAST2 (Altschull and Gish, Meth. Enzymol. 266:460-480 (1996)), FASTA (Pearson, Meth. Enzymol. 266:227-258 (1996)), or SSEARCH (Pearson, supra) to identify regions of homology. One skilled in the art can also identify regions of potential similarity using an algorithm that compares the encoded polypeptide structure. Such algorithms include, for example, SCOP, CATH, or FSSP which are reviewed in Hadley and Jones, Structure 7:1099-1112 (1999). Additionally, hybridization kinetics, specificity and annealing conditions are similarly well known in the art. These and other nucleic acid characteristics, hybridization methods and annealing conditions useful for specifically identifying a complementary sequence within high or low complexity samples are similarly well known in the art. Further, annealing conditions sufficient for high, moderate or low stringency hybridization also is well known in the art. These and other methods can be found described in, for example, Sambrook et al., Molecular Cloning: A Laboratory Manual, Cold Spring Harbor Laboratory, New York (1992), and in Ausebel et al., Current protocols in Molecular Biology, John Wiley and Sons, Baltimore, Md. (2000).

Generally, mutagenic primers, PAPs and SAPs utilized in combinatorial mutagenesis will consist of synthetic oligonucleotides but can consist of any nucleic acid sequence having sufficient complementarity to specifically anneal to the target parental nucleic acid for primer-directed polymerase extension. Synthetic oligonucleotides can be routinely designed and synthesized with high efficiency and yield. Methods for the synthesis of oligonucleotides including, for example, DNA, RNA analogues and modified forms thereof are well known in the art. Such methods can be found described in, for example, Oligonucleotide Synthesis: A Practical Approach, Gate, ed., IRL Press, Oxford (1984). Synthesis of oligonucleotides can be accomplished using both solution phase and solid phase methods. Solid phase oligonucleotide synthesis employs mononucleoside phosphoramidite coupling units and involves reiteratively performing four steps of deprotection, coupling, capping, and oxidation as has been described, for example, by Beaucage and Caruthers, Tetrahedron Letters 22: 1859-1862 (1981). Oligonucleotide synthesis via solution phase can be accomplished with several coupling mechanisms, and can include, for example, the use of phosphorous to prepare thymidine dinucleoside and thymidine dinucleotide phosphorodithioates. Methods useful for preparing oligonucleotides via solution phase are well known in the art and described by Sekine et. al., J. Org. Chem. 44:2325 (1979); Dahl, Sulfer Reports, 11:167-192 (1991); Kresse et al., Nucleic Acids Res. 2:1-9 (1975); Eckstein, Ann. Rev. Biochem., 54:367-402 (1985); and Yau, U.S. Pat. No. 5,210,264.

Synthesis or amplification of a first product having a first mutagenized portion can proceed by annealing a first mutagenic primer and a first PAP to a parental nucleic acid. As described previously, the mutagenic primer and PAP anneal to noncontiguous regions of the parental nucleic acid. Once annealed, the trimolecular hybridization complex will consist of a parental nucleic acid annealed to an upstream mutagenic primer and a downstream PAP. The upstream mutagenic primer will contain imperfect base pairing where the non-complementary nucleotides correspond to the altered bases that are to be incorporated into the first product. Extending one or both of the annealed primers by, for example, enzymatic polymerization will product a first product that is a fragment of the parental nucleic acid. Through incorporation of the mutagenic primer and the PAP, the first product will contain the altered bases or mutations designed into the mutagenic primer. These mutations will reside in the upstream mutagenic region of the parental nucleic acid fragment. The first product also will contain at its downstream terminus the unique sequence tag incorporated through the PAP.

Bidirectional extension directed from the use of two primers such as a mutagenic primer and a PAP will inherently result in exponential amplification of the first product. This result will occur because synthesis occurs from both strands of the template. The first products synthesized can be employed directly in subsequent iterations of combinatorial mutagenesis. Alternatively, such first products can be further amplified prior to use in subsequent iterations. Additional amplification can be performed, for example, through PCR and theromocycling procedures to increase yield of the first product.

The first product also can be subjected to additional procedures to increase specificity of subsequent iterations and, consequently, overall production of final modified products containing the various designed mutations. For example, another result that can occur through the bidirectional amplification of the parental nucleic acid is production of full length or “long” product derived from extension of the PAP. Synthesis of long products is shown in FIG. 2A where a downstream PAP directs polymerase extension to the opposite terminus of the parental nucleic acid. To reduce background noise and therefore increase specificity of the amplification steps, various procedures can be employed to selectively remove undesirable long products from the reaction mixture. Such procedures include for example size fractionation, gel electrophoresis and fragment isolation as well as other methods well known to those skilled in the art.

An efficient alternative to removal of long products that employ an additional step can be performed by selectively destroying long products. In this regard, selective destruction can be performed simultaneously or consecutively in the same reaction mixture without the need for additional isolative manipulations. Numerous procedures can be employed for the selective destruction of long products over the amplified first products. Similarly, selective destruction or template inactivation also can be performed at the analogous step in subsequent iterations of the combinatorial mutagenesis methods of the invention.

Selective destruction can be performed by, for example, treating the mixture containing the first amplified product having a first mutagenized portion with a cleaving reagent selective for a sequence in the parental nucleic acid that is absent in the amplified product. Cleaving reagents applicable for selective destruction include, for example, restriction endonucleases where the cleavage recognition site is present in the long product but not in the first product having a mutagenized portion. FIG. 2B exemplifies the use of a Dpn I restriction enzyme that selectively destroys long products.

DpnI digests methylated double-stranded plasmid DNA derived from most E. coli strains. “Long product” is single-stranded DNA which is derived from a PAP which primes to and is extended from the original parental plasmid (or double-stranded PCR product) DNA. DpnI does not digest single-stranded DNA. Digestion of parental double stranded DNA occurs after the synthesis with the mutagenic primer and before addition of a PAP. The rationale being that PAPs can only prime the nascent single-stranded mutagenic DNA and long products don't have a chance to form since the paternal template is destroyed. Any restriction enzyme (preferably a frequent cutter) can be used to digest parental DNA whether of plasmid or PCR product forms. Digestion, therefore, is to prevent the formation of rather than destroy long product. Although a unique site can be exploited in certain instances to selectively digest long products.

Essentially any restriction enzyme can be used so long as the recognition site is present in the long product but absent in the mutagenized first product. Similarly, in subsequent iterations, the restriction recognition site would be present in each respective long product but absent in the mutagenized second or tertiary products. Cleaving reagents other than restriction enzymes well known to those skilled in the art also can be employed to selectively destroy long products over the desired mutagenized first, second or tertiary products. Such other cleaving reagents can include, for example, chemical cleavage, affinity cleavage reagents and photoaffinity cleavage reagents.

Following removal or destruction of long products from the mixture containing a first product having a first mutagenized portion containing one or more altered nucleotides, the mutagenized products can be purified for storage or subsequent use. Alternatively, the first products or the reaction mixture containing first products can be annealed to the parental nucleic acid and used as a primer for an amplification reaction to recombine the first mutagenized product with the remainder of the parental nucleic acid. Such a recombination step results in reconstruction of the parental nucleic acid sequence with the inclusion of the primer directed mutations and the unique sequence tag incorporated via the PAP. Accordingly, the product of a first recombination step in the combinatorial mutagenesis methods of the invention corresponds to a first modified parental nucleic acid containing a first mutagenized portion.

Sequential, parallel or multiplex combinatorial mutagenesis allows the generation of populations of first mutagenized products and first modified parental nucleic acids containing predetermined mutations. As described further below, such populations can be small, medium, large or highly diverse. Also as described further below, use of the first modified parental nucleic acids in subsequent iterations similarly allows for the generation of a wide range of population sizes of secondary or tertiary modified parental nucleic acids, including small, medium, large or highly diverse populations. Such secondary or tertiary modified parental nucleic acids will exhibit, for example, two or more mutagenized regions. Each modified parental nucleic acid will harbor two or more predetermined nucleotide alterations predesigned and implemented through their respective mutagenic primers. Identification of each species of first, second or tertiary modified parental nucleic acid can be identified using the associated unique sequence tag.

First modified parental nucleic acids can be used directly in subsequent iterations of the combinatorial mutagenesis methods of the invention or, alternatively, they can be isolated and subsequently employed in further iterations. Additionally, either the mixture or isolated first modified parental nucleic acids can be stored for later use as convenient, or for use in the same or a different combinatorial mutagenesis schemes. Procedures for storage and subsequent use of nucleic acids or nucleic acid polypeptide mixtures are well known to those skilled in the art and can be found described in, for example, Sambrook et al., supra, (1992), and in Ausebel et al., supra, (2000).

Iterative rounds of combinatorial mutagenesis can be carried out with or without separate isolation procedures or other manipulations of the first modified parental nucleic acids. Added efficiency can be achieved by omitting a separate isolation step and directly using a first, second or any tertiary modified parental nucleic acid in subsequent iterations. Amplification of first modified parental nucleic acids can be accomplished, for example, via PCR or other linear or exponential procedure to increase the amount of primer sequence available for subsequent rounds of mutagenesis. Amplification using PCR or other primer directed polymerization can occur using a second PAP complementary to a region upstream from the mutagenic region and a SAP complementary to the unique sequence tag associated with the first PAP and downstream of the mutagenic region. Employing a SAP complementary to the sequence tag maintains the linkage of the unique sequence tag and the mutations incorporated in the first mutagenic product. The second PAP can correspond to any region of the parental nucleic acid sequence upstream of the mutagenic region.

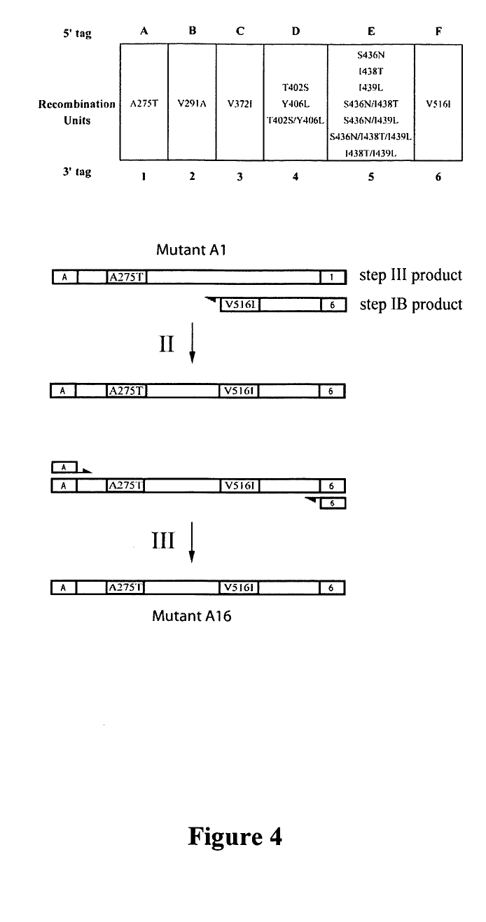

For the duplication of a complete copy of the recombined sequence corresponding to the parental nucleic acid with incorporated mutations, the second PAP will generally correspond to the upstream terminus of the parental nucleic acid. Additionally, as shown in FIG. 4, the second PAP also can contain, for example, a further unique sequence tag which is specifically associated with the mutagenic sequence. Using both upstream and downstream unique sequence tags in the combinatorial mutagenesis methods of the invention allows for a hierarchical classification system to index, organize and identify each different species within a diverse population of modified parental nucleic acids. Recombination using a first product is shown in FIG. 1, step II with reference to a crossover product. Recombination using mutagenic products such as that shown in FIG. 2A or 2B is performed similarly except that a first product having a mutagenized portion is employed as the primer instead of a crossover product. Selective amplification and iteration as shown in FIG. 1 also is performed similarly with the substitution of a first mutagenized portion in place of a crossover product.

Additional mutations can be incorporated into the parental nucleic acid sequence by, for example, repeating the above steps employing the product from the final amplification as the parental template in the next iteration of combinatorial mutagenesis. For example, first modified parental nucleic acids obtained following amplification employing a second PAP and a SAP primer pair can be annealed with a second mutagenic primer and a third PAP. As with the first mutagenic primer and first PAP pair, the second mutagenic primer and third PAP pair anneal to noncontiguous regions of the first modified parental nucleic acid, which is employed as a parental nucleic acid in such subsequent iterations. The third PAP also is associated with a unique sequence tag that identifies the incorporated mutations from the second mutagenic primer.

Once annealed, the primers can be extended by enzymatic polymerization for synthesis or amplification of the annealed primer pairs for each modified parental nucleic acid within a set, population or mixture, to generate a second product having a second mutagenized portion. The steps of treating the product with a cleaving reagent selective for long products, recombination with the parental nucleic acid by annealing and extension of the second mutagenized product to the parental nucleic acid will produce a second modified parental nucleic acid containing a first mutagenized portion and at least one second mutagenized portion. As described previously, subsequent iterations of the combinatorial mutagenesis methods of the invention, the parental nucleic acid will correspond, for example, to the modified parental nucleic acid obtained in the one or more of the preceding rounds of mutagenesis. Additionally, the second modified parental nucleic acid also can be amplified employing a SAP specific to the unique sequence tag associated with the third PAP.

Employing the product of a predecessor iteration as the starting parental nucleic acid template in a subsequent iteration allows for the sequential incorporation of additional defined mutations into the parental nucleic acid. Accordingly, subsequent iterations of the combinatorial mutagenesis methods of the invention can be performed using tertiary mutagenic primers and PAP pairs, recombined and amplified with SAPs corresponding to each unique sequence tag associated with the tertiary PAPs. As will be understood by those skilled in the art given the teachings and guidance provided herein, the number of iterations is limited only by the size of the initial parental nucleic acid. Accordingly, diverse populations of mutagenized parental nucleic acids having predetermined and controlled sequence and mutational compositions can be efficiently synthesized through serial, parallel or multiplex application of the steps above.

The combinatorial mutagenesis methods of the invention allow for the creation of variant nucleic acids of essentially any designed sequence change or combination of sequence changes compared to a parental nucleic acid. Additionally, the combinatorial mutagenesis methods of the invention also allow for the creation of essentially any designed sequence change or combination of sequence changes between different parental nucleic acids or compared to multiple parent nucleic acids. The resultant variant nucleic acids, corresponding to first, second or tertiary modified parental nucleic acids, can be produced to have one or many changed residues. Accordingly, the number of mutations that can be incorporated can include, for example, 1, 2, 3, 4, 5, 10, 15, 20, 25 or more mutations and include all possible changes and combination of changes within a portion of a parental nucleic acid. Additionally, the number of mutations that can be incorporated can include, for example, all possible of changes and combination of changes within the entire sequence of a parental nucleic acid. Parental nucleic acids can be, for example, small, medium or large.

First, second and tertiary modified parental nucleic acids also can be produced to have one or many mutagenized portions containing one or more mutations in each mutagenized portion compared to a parental nucleic acid, multiple parental nucleic acids or between different parental nucleic acids. Accordingly, the number of mutagenized portions that can be incorporated into first, second or tertiary modified parental nucleic acids can include, for example, 1, 2, 3, 4, 5, 10, 15, 20, 25 or more depending on the size of the parental nucleic acid and the chosen size of a portion to be modified. All possible combinations and permutations of mutagenized portions can be designed and produced as well as the introduction of partially or completely mutagenized portions spanning the entire length of a parental nucleic acid. Additionally, the combinatorial mutagenesis methods of the invention can be used to design and produce from one to many mutations in some or all mutagenized portions. For example, one mutagenized portion in a second or tertiary modified parental nucleic acid can contain one or a few mutations while another mutagenized portion in the same second or tertiary modified parental nucleic acid can contain many to all possible mutations. Additionally, all possible combinations or permutations of from one to all possible mutations incorporated in different mutagenized portions also can be designed and produced using the combinatorial mutagenesis methods of the invention.

Changes can be designed with respect to the primary nucleotide sequence or with respect to the encoded nucleic acid. For example, from one to hundreds or more different nucleotide changes can be designed and produced compared to a parental nucleic acid. Alternatively, from one to hundreds or more different codon changes, encoding from one to hundreds or more different amino acids, can be designed and produced using the combinatorial mutagenesis methods of the invention. Accordingly, the combinatorial mutagenesis methods of the invention can produce first, second or tertiary modified parental nucleic acids encoding, for example, 1, 2 or 3 or more amino acid changes. First, second or tertiary modified parental nucleic acids encoding, for example, between about 3-25 or between about 4-20 amino acid changes as well as all ranges or integer values above, below or within these ranges can be designed and efficiently produced using the combinatorial methods of the invention. Therefore, second or tertiary modified parental nucleic acids can be produced that encode from 2-500, greater than 500, between about 3-10⁴, between about 26-10³ or greater than about 10⁴ amino acid changes. Given the teachings and guidance provided herein, those skilled in the art will know how to design mutational variants of parental nucleic acids with a few or with many mutations or either at the nucleotide level or at the codon level to produce variant gene products.

Various strategies can be implemented to design and produce first, second or tertiary modified nucleic acids of the invention. For example, strategies can employ mutagenic primers that direct site-specific changes of defined nucleotides at one or more positions, including all positions within the mutagenic region of the primer. In this regard, the mutagenic primers are designed to incorporate predetermined changes at one or more specific positions. The changes can be designed at the nucleotide level or at the codon level to alter an encoded amino acid residue. Mutagenic primers can be designed to contain flanking sequences sufficiently complementary to the parental nucleic acid sequences flanking regions to allow annealing and subsequent incorporation of the mutated bases. The use of mutagenic primers for site directed changes can be beneficial to produce discrete populations of variants of defined composition. Such populations can be small, large or highly diverse using the combinatorial methods of the invention.

Another strategy can employ mutagenic primers with random nucleotide sequences to produce a diverse number of changes in the parental nucleic acid. For example, the mutagenic region of the primer can contain an N at one or more positions where N consists of a mixture of the four nucleotides A (adenine), T (thymine), G (guanine) and C (cytosine). Various ratio of some or all of the four nucleotides also can be employed. The use of different ratios can be particularly useful to alter encoded amino acid sequences by changing the corresponding codon sequence. For example, mutagenic primers can be used that direct codon changes using a partially degenerate codon sequence such as NNK where N corresponds to equal molar ratio of A, T, G and C, and K corresponds to an equal molar ratio of G and T. The use of partially degenerate codons reduces redundancy of the genetic code from 64 to 32. Various other ratios of nucleotides also can be incorporated at one or more positions of the mutagenic primer to produce desired and predetermined ratios. For example, nucleotide ratios can be used to generate variegated codons such as that described in U.S. Pat. No. 5,223,409. Variegated codon synthesis allow for the generation of a wide range of codon frequencies via incorporation of different nucleotide ratios in the encoding nucleic acid. These and other synthesis methods are well known in the art for mutagenesis of nucleotide sequences or their encoding amino acids. Given the teachings and guidance provided herein, it will be apparent to those skilled in the art that these methods as well as others will known in the art can be utilized for directing mutational changes at any predetermined position in a parental nucleic acid. Such changes can be a single nucleotide or ratios of some or all nucleotides to produce some or all possible changes at a particular position.

In addition to adjusting a nucleotide format incorporated into a mutagenic primer, various other mutagenic primer designs can be employed to augment, for example, diversity of resultant populations or the efficiency of the combinatorial mutagenesis methods of the invention. Diversity can be increased by, for example, increasing the number of changes or mutagenic regions harbored in a mutagenic primer. The greater the number of mutations harbored in a mutagenic primer the more changes can be introduced in the same number of steps. Similarly, a primer can have both mutagenic positions or regions as well as complementary positions or regions compared to the parental nucleic acid such that a single mutagenic primer directs mutations at multiple non-adjacent regions within a selected mutagenic region. Such primers are termed herein as bridging oligonucleotides and can contain, for example, one, two, three, four or five or more different mutagenic or complementary positions or regions.

Additional primer strategies also can be implemented that include other mutagenesis methods. For example, chimeric primers such as those used in SCOPE can be utilized to generate hybrid molecules. The chimeric primers can be used alone or in combination with the mutagenesis primers of the invention. Other combinations or permutations of primer strategy or mutagenesis method well known in the art also can be employed together with the combinatorial mutagenesis methods of the invention.

Additional strategies for design and implementation also can be employed for generating modified parental nucleic acids of the invention. Such strategies include, for example, combinatorial mutagenesis by sequential order of the steps described previously. Any of the above strategies also can be implemented by separately generating the various designed modified parental nucleic acids so that individual species of a resultant population exists separately.

Alternatively, the combinatorial mutagenesis methods of the invention can be implemented employing a number of other formats to efficiently generate a resulting population where, for example, all species are produced in a combined mixture or pools of combined mixtures. Individual nucleic acid species within such populations can then be identified by, for example, their associated unique sequence tags. Such other formats include, for example, the serial, parallel or multiplex mutation incorporation, amplification of first, second or tertiary products, destruction of long products, recombination with parental nucleic acid to produce first, second or tertiary modified nucleic acid and iteration.

Serial formats include step-wise progress through the above steps. Parallel formats include step-wise or multiplex progress through the above steps where different parental nucleic acids can be involved or where different steps are occurring separately but together with other combinatorial mutagenesis reactions. Multiplex formats include the simultaneous occurrence of two or more combinatorial mutagenesis steps in the same reaction vessel or simultaneous occurrence of two or more combinatorial mutagenesis processes occurring in the same reaction vessel, such as when two or more parental nucleic acids are being changed simultaneously. Other formats well known in the art can similarly be employed in the methods of the invention. Similarly, any combination of serial, parallel or multiplex format also can be employed to achieve the variant populations of the invention.

Therefore, the invention provides a method for the combinatorial mutagenesis of a parental nucleic acid. The method consists of: (a) extending by enzymatic polymerization a plurality of first mutagenic primers and a plurality of first PAPs annealed to noncontiguous regions of a parental nucleic acid to produce a mixture containing a plurality of first products each having a first mutagenized portion comprising one or more altered nucleotides, each of the plurality of first PAPs containing a unique sequence tag associating mutations within each of the first mutagenic primers with the plurality of first PAPs; (b) treating the plurality of first extension products or first products with a cleaving reagent selective for a nucleotide sequence present in the parental nucleic acid but absent in the plurality of first products; (c) annealing the plurality of first products to the parental nucleic acid, and (d) extending by enzymatic polymerization the annealed plurality of first products to produce a plurality of first modified parental nucleic acids containing a first mutagenized portion. The plurality of first extension products or first products treated with the cleaving reagent also can be amplified.

The method of combinatorial synthesis can further include the step: (e) amplifying the plurality of first modified parental nucleic acids containing a first mutagenized portion by polymerase extension of an annealed plurality of first SAPs to the unique sequence tag contained in the plurality of first PAPs and an annealed plurality of second PAPs to the first modified parental nucleic acid, the plurality of first and second PAPs corresponding to opposite termini of the parental nucleic acid. The method can additionally include step (f), consisting of repeating steps (c) through (d) or steps (c) through (e) one or more times by annealing the plurality of first products produced in step (a) to the plurality of first modified parental nucleic acids produced in step (d) to generate a plurality of second modified parental nucleic acids containing a first mutagenized portion and at least one second mutagenized portion. Further, the method of combinatorial mutagenesis also can include step (i) repeating step (f) at least once by annealing the plurality of first products to the plurality of first or second modified parental nucleic acids and a plurality of tertiary PAPs to generate a plurality of tertiary modified parental nucleic acids containing first, second and tertiary mutagenized portions.

The invention provides a hierarchical classification system associating sequences between a mutagenic and a noncontiguous parental region of a nucleic acid. The system consists of: (a) a recombination matrix indexing a plurality of 5′ and 3′ unique sequence tags associated with a plurality of mutagenic primer sequences, the indexing relating a 5′ unique sequence tag, one or more mutagenic sequences and a 3′ unique sequence tag, wherein a 5′ or a 3′ unique sequence tag identifies a mutagenic sequence incorporated into a parental nucleic acid sequence, and wherein both 5′ and 3′ unique sequence tags identify a combination of mutagenic sequences incorporated into a parental nucleic acid.

The mutagenic methods of the invention can be used to generate small, medium, large or highly diverse populations of modified parental nucleic acids. As described previously, particular variants within such populations can be identified using the unique sequence tags associated with each PAP. In the specific instance, where a first modified parental nucleic acid contains a single mutation or mutagenized portion, the first modified parental nucleic acid can contain a unique sequence tag at either its 5′ or 3′ terminus. The first modified parental nucleic acid also can contain a different unique sequence at each of its termini. In either instance, amplification with a SAP corresponding to the either or both of the unique sequence tags will generate a product having the associated mutation or mutagenic portion. Similarly, whether a first, second or tertiary modified nucleic acid contains one, two, three, four or five or more mutations or mutagenic regions, for example, the same utilization of unique tags and SAPs can be employed to identify single, multiple or all modified parental nucleic acids in a resulting combinatorial mutagenesis population.

In instances where the combinatorial mutagenesis products result from iterative rounds and contain more than one mutation or mutagenic portion, organization and utilization of unique sequence tags in a hierarchical classification system can facilitate identification of any modified parental nucleic acid species generated in the population. One hierarchical classification system that can be used is shown in FIG. 4. This scheme utilizes a recombination matrix that associates 5′ and 3′ unique sequence tags with a particular mutation or mutagenic region incorporated into a parental nucleic acid.

Briefly, a recombination matrix indexes a plurality of 5′ and 3′ unique sequence tags with each of their respective associated mutagenic primer sequence. The matrix therefore provides a one to one index of 5′ and 3′ unique sequence tags to an associate mutagenic sequence. Both 5′ and 3′ unique sequence tags will generally be associated with full-length mutagenic products compared to a parental nucleic acid sequence, or compared to the complete region of a parental nucleic acid sought to be mutagenized when such a region corresponds to a less than full-length sequence. Accordingly, a matrix of the invention will show correlations of, for example, both 5′ and 3′ unique sequence tags associated with first, second and tertiary modified parental nucleic acids of the invention.

For example, a modified parental nucleic acid having the first mutation shown in FIG. 4 also will have associated with it a 5′ tag A and a 3′ tag 1. Any sequence amplified using SAPs corresponding to A and 6 will have the corresponding first mutation shown in, for example, FIG. 4. Another specific example is the second modified parental product resulting from the combinatorial mutagenesis of the sequence shown in FIG. 4. Two mutations are shown incorporated at the bottom of FIG. 4. One mutation resulting from a first combinatorial mutagenesis iteration is associated with a 5′ tag A while another mutation resulting from a second combinatorial mutagenesis iteration is associated with a 3′ tag 6. The resultant product, corresponding to a second modified parental nucleic acid therefore contains a 5′ tag A and a 3′ tag 6 which indicate that both corresponding mutations indexed to these tags in the recombination matrix are present in the mutagenic nucleic acid product.

The matrix similarly provides a one to one index of 5′ or 3′ unique sequence tags to an associated mutagenic sequence where the mutagenic product is less than a full-length sequence compared to the parental nucleic acid sequence or the complete region sought to be mutagenized. Accordingly, a matrix of the invention will show correlations of, for example, a 5′ or a 3′ unique sequence tag associated with first, second or tertiary products of the invention. For example, a first product generated for producing a modified parental nucleic acid having the first mutation shown in FIG. 4 will have associated with it a 5′ tag A or a 3′ tag 1. Similarly, a first product generated for producing a modified parental nucleic acid having the sixth mutation shown in FIG. 4 will have associated with it a 5′ tag F or a 3′ tag 6. Exemplified in FIG. 4 is a first product having a 3′ tag 6. Use of this first product for incorporating the shown sixth mutation also will incorporate the associated 3′ tag 6 as shown. Any sequence amplified using SAPs corresponding to 6 will have the corresponding sixth mutation shown in, for example, FIG. 4.

Design and application of a recombination such that there is a one to one correspondence between a mutagenic sequence and 5′, 3′ or both 5′ and 3′ unique sequence tags allows for the incorporation and subsequent identification of specified mutations into a parental nucleic acid. The matrix provides a cross-reference of which mutations are associated with a particular tag. Therefore, by identifying the tag or tags associated with a modified product, one can concurrently identify the incorporated mutations in the modified product. Iterations of the combinatorial mutagenesis methods of the invention will combine unique sequence tags into resultant products just as their associated mutations are similarly combined into a single nucleic acid sequence. When combined, hybrid associations between 5′ and 3′ tags and mutations will be formed. These hybrids will therefore identify the mutational combinations and the nomenclature derived from the matrix will describe then as such. The modified parental nucleic acid A16 shown in FIG. 4 is an example of a matrix nomenclature that identifies a two mutation combination.

Essentially any number of associations between mutations and 5′ or 3′ unique sequence tags can be indexed in a recombination matrix of the invention. Similarly, a recombination matrix also can be used to identify a modified parental nucleic acid containing an essentially unlimited number of mutations. Exemplification of a recombination matrix has been described above and shown in FIG. 4 with reference to incorporation of two mutations into a parental nucleic acid sequence. However, given the teachings and guidance provide herein, those skilled in the art will understand that by the nomenclature of combined sequence tags will identify more than two mutations in a single nucleic acid. Moreover, the hierarchical classification of the invention also can use, for example, different or multiple recombination matrices for different iterations or for different parental nucleic acids or a combination of both. For example, as the number of mutations or the number of iterations increases, it can be beneficial to employ a different recombination matrix with a different iteration or in association with a different parental nucleic acid sequence. Therefore, the associations required from a recombination matrix can therefore be present in the same or different matrices so long as such associations index a unique 5′ and a unique 3′ tag with a mutagenic sequence.

The design of a recombination matrix entails the indexing of 5′ and 3′ unique sequence tags to an associated mutagenic sequence. The matrix shown in FIG. 4 is one format that can be employed. However, essentially any format that associates 5′ and 3′ unique sequence tags with a mutagenic sequence is applicable for use as a recombination matrix of the invention. Such formats can directly or indirectly associate 5′ and 3′ tags with a mutagenic sequence. Once the indexed associations are formed in a recombination matrix, a user can link a unique sequence tag with a PAP and employ it with a corresponding mutagenic primer. The unique tag, or combinations of unique tags such as that described above will therefore identify mutations incorporated into a parental nucleic acid.

Identification can be performed by essentially any method well known to those skilled in the art that can detect a unique sequence. Such methods include nucleic acid hybridization. Specific hybridization of a probe to a unique sequence tag or to multiple unique sequence tags incorporated to a modified parental nucleic acid sequence will identify the mutational variations associated with the unique tags. Various hybridization methods and methods based on hybridization well known in the art are applicable for specific detection of a unique sequence tag. For example, linear amplifications such as primer extension or exponential amplifications including PCR and ligase chain reaction can be employed using SAPs specific to the unique sequence tags. Other methods well known in the art also can be employed. Hybridization, amplification and other methods well known in the art utilizing hybridization as a means for identification or specificity can be found described in, for example, Sambrook et al., supra, (1992), and in Ausebel et al., supra, (2000).

A further modification of the indexing system can include the use of tertiary amplification primers (TAPs). These primers contain sequence at their 3′ ends that correspond to a given SAP and have unique sequence at their 5′ end. TAPs can be used to provide additional information about the combination of mutation in the encoded gene at later iterations in the recombination process.

Other methods well known in the art for detection and specific identification also can used in the methods of the invention. In this regard, unique tags can be incorporated into PAPs that are detectable by, for example, radiation, fluorescence, phosphorescence, luminescence or enzyme activity. Different labels can be covalently attached to a PAP or to a SAP and then employed similarly to the hybridization protocols described. Measurement of a signal produced from the detectable label will identify the associated modified parental nucleic acid. Unique detectable labels and methods of detection are well known in the art. Given the teachings and guidance provided herein, those skilled in the art will know understand how to substitute detectable labels or methods other than hybridization for the unique sequence tags and primer mediated nucleic acid hybridizations and amplification reactions described herein. For example, it will be understood that so long as there is a correspondence between a unique label, an incorporated mutation and a detection method available for the unique label, then a particular modified parental nucleic acid within a mixture or population of modified parental nucleic acid products can be readily identified or isolated.

Nucleic acid amplification methods can be particularly useful for identification of modified parental nucleic acid sequences. Such methods offer the specificity and flexibility of nucleic acid hybridization and also increase the copy number of the target nucleic acid. Moreover, procedures such a PCR offer the advantage of bidirectional amplification which allows further flexibility in indexing a unique sequence tag to a mutagenic sequence. The use of two primers for bidirectional primer extension further amplifies the product in an exponential manner, allowing for a smaller number of reactions to generate sufficient product for either the next iterative cycle of the combinatorial mutagenesis methods of the invention or for detection and identification of the desired first, second or tertiary modified parental nucleic acid.

Detection or identification of desired modified parental nucleic acids can be performed by specifically annealing 5′ and 3′ SAPs to the modified parental nucleic acid and amplifying it through one or more cycles of primer extension or PCR. The modified parental nucleic acid can be within a population of modified nucleic acids obtained following combinatorial mutagenesis. Specific hybridization of the SAPs to unique sequence tags associated with the modified parental nucleic acids will result in the specific or preferential amplification of the desired variant over other sequences within the population. The amplified modified parental nucleic acid can be isolated or cloned into a vector for subsequent manipulations or expressed to synthesis the encoded polypeptide. Methods for annealing and conditions for specific hybridization are well known in the art and can be found described in, for example, Sambrook et al., supra, (1992), and in Ausebel et al., supra, (2000).

The methods described above for identifying a desired modified parental nucleic acid can be used to identify any variant sequence designed and synthesized using the combinatorial mutagenesis methods of the invention. Moreover, using unique sequence tags and a recombination matrix that indexes the tags to their associated mutagenic sequences allows simplification or deconvolution of both simple or complex populations of modified parental nucleic acids. The simplification can be achieved by, for example, identifying the individual parts of the mixture or population of modified parental nucleic acids. Identification of individual species within a population can occur as routinely as the identification of multiple species or all species within a population of modified nucleic acids. Therefore, the methods described above can be employed to deconvolute one, some or all modified parental nucleic acids within a population.

Because deconvolution involves the identification of the individual modified parental nucleic acids, and therefore, and therefore employs the specificity of unique sequence tags, the process can be performed in either serial, parallel or multiplex formats. The process also can be performed in various combinations of these formats. For example, a single pair of SAPs can be employed to identify a particular species within the population. Alternatively, all pairs of SAPs corresponding to all of their associated modified parental nucleic acids can be employed, for example, in a single reaction, or multiplex format; in multiple reactions, or parallel formats, or each pair in an individual reaction, or serial format. The specificity of unique sequence tags and hybridization methods are particularly beneficial for rapid and efficient deconvolution of populations in multiplex formats.

Therefore, the invention provides a method of deconvoluting a plurality of mutations introduced into a parental nucleic acid sequence. The method consists of: (a) forming a recombination matrix indexing a plurality of 5′ and 3′ unique sequence tags to a mutagenic primer sequence; (b) amplifying a plurality of modified parental nucleic acid sequences having a plurality of incorporated mutations associated with one or more unique sequence tags corresponding to 5′, 3′ or both 5′ and 3′ noncontiguous regions compared to a region of complementarity to the mutagenic primer, the amplification using a pair of SAPs corresponding to the unique sequence tags, and (c) correlating the amplification products obtained with each SAP of the pair of SAPs to its associated mutagenic primer sequence to identify the plurality of incorporated mutations within a modified parental nucleic acid sequence.

Once the populations of modified parental nucleic acids have been constructed as described above, they can be expressed to generate a population of variant polypeptides that can be screened for a desired activity. Alternatively, individually identified modified parental nucleic acids can be isolated and expressed to produce the encoded variant polypeptide. The activity screened for can be the same activity exhibited by its parental polypeptide. Alternatively, individual or populations of expressed variant polypeptides can be screened for an activity different from that exhibited by a parental polypeptide.

For example, the nucleic acids encoding the changed polypeptides can be cloned into an appropriate vector for propagation, manipulation and expression. Such vectors are known or can be constructed by those skilled in the art and should contain all expression elements sufficient for the transcription, translation, regulation, and if desired, sorting and secretion of the variant polypeptide or polypeptides. The vectors also can be for use in either procaryotic or eukaryotic host systems so long as the expression and regulatory elements are of compatible origin. The expression vectors can additionally included regulatory elements for inducible or cell type-specific expression. One skilled in the art will know which host systems are compatible with a particular vector and which regulatory or functional elements are sufficient to achieve expression of a polypeptide in soluble, secreted or cell surface forms.

Suitable expression vectors are well-known in the art and include vectors capable of expressing nucleic acid operatively linked to a regulatory sequence or element such as a promoter region or enhancer region that is capable of regulating expression of such nucleic acid. Promoters or enhancers, depending upon the nature of the regulation, can be constitutive or inducible. The regulatory sequences or regulatory elements are operatively linked to a nucleic acid of the invention or population of first, second or tertiary modified parental nucleic acids as described above in an appropriate orientation to allow transcription of the nucleic acid.

Appropriate expression vectors include those that are replicable in eukaryotic cells and/or prokaryotic cells and those that remain episomal or those which integrate into the host cell genome. Suitable vectors for expression in prokaryotic or eukaryotic cells are well known to those skilled in the art as described, for example, in Ausubel et al., supra. Vectors useful for expression in eukaryotic cells can include, for example, regulatory elements including the SV40 early promoter, the cytomegalovirus (CMV) promoter, the mouse mammary tumor virus (MMTV) steroid-inducible promoter, Moloney murine leukemia virus (MMLV) promoter, and the like. A vector useful in the methods of the invention can include, for example, viral vectors such as a bacteriophage, a baculovirus or a retrovirus; cosmids or plasmids; and, particularly for cloning large nucleic acid molecules, bacterial artificial chromosome vectors (BACs) and yeast artificial chromosome vectors (YACs). Such vectors are commercially available, and their uses are well known in the art. One skilled in the art will know or can readily determine an appropriate promoter for expression in a particular host cell.

Appropriate host cells, include for example, bacteria and corresponding bacteriophage expression systems, yeast, avian, insect and mammalian cells and compatible expression systems known in the art corresponding to each host species. Methods for recombinant expression of populations of progeny polypeptides or progeny polypeptides within such populations in various host systems are well known in the art and are described, for example, in Sambrook et al., supra and in Ansubel et al., supra. The choice of a particular vector and host system for expression and screening of progeny polypeptides will be known by those skilled in the art and will depend on the preference of the user. For example, expression systems for soluble polypeptides either cytoplasmically or extracellularlly are well known in the art. Similarly, surface expression on bacteriophage, prokaryotic and eukaryotic cells is similarly well known in the art.

The recombinant cells are generated by introducing into a host cell a vector or population of vectors containing a nucleic acid molecule encoding a polypeptide. The recombinant cells are transducted, transfected or otherwise genetically modified by any of a variety of methods known in the art to incorporate exogenous nucleic acids into a cell or its genome. Exemplary host cells that can be used to express a polypeptide include mammalian primary cells; established mammalian cell lines, such as COS, CHO, HeLa, NIH3T3, HEK 293 and PC12 cells; amphibian cells, such as Xenopus embryos and oocytes; and other vertebrate cells. Exemplary host cells also include insect cells such as Drosophila, yeast cells such as Saccharomyces cerevisiae, Saccharomyces pombe, or Pichia pastoris, and prokaryotic cells such as Escherichia coli.

In one embodiment, a nucleic acids encoding a polypeptide can be delivered into mammalian cells, either in vivo or in vitro using suitable vectors well-known in the art. Suitable vectors for delivering a nucleic acid encoding a polypeptide to a mammalian cell, include viral vectors such as retroviral vectors, adenovirus, adeno-associated virus, lentivirus, herpesvirus, as well as non-viral vectors such as plasmid vectors.

Viral based systems provide the advantage of being able to introduce relatively high levels of the heterologous nucleic acid into a variety of cells. Suitable viral vectors for introducing a nucleic acid encoding a polypeptide into mammalian cells are well known in the art. These viral vectors include, for example, Herpes simplex virus vectors (Geller et al., Science, 241:1667-1669 (1988)); vaccinia virus vectors (Piccini et al., Meth. Enzymology, 153:545-563 (1987)); cytomegalovirus vectors (Mocarski et al., in Viral Vectors, Y. Gluzman and S. H. Hughes, Eds., Cold Spring Harbor Laboratory, Cold Spring Harbor, N.Y., 1988, pp. 78-84)); Moloney murine leukemia virus vectors (Danos et al., Proc. Natl. Acad. Sci. USA, 85:6460-6464 (1988); Blaese et al., Science, 270:475-479 (1995); Onodera et al., J. Virol., 72:1769-1774 (1998)); adenovirus vectors (Berkner, Biotechniques, 6:616-626 (1988); Cotten et al., Proc. Natl. Acad. Sci. USA, 89:6094-6098 (1992); Graham et al., Meth. Mol. Biol., 7:109-127 (1991); Li et al., Human Gene Therapy, 4:403-409 (1993); Zabner et al., Nature Genetics, 6:75-83 (1994)); adeno-associated virus vectors (Goldman et al., Human Gene Therapy, 10:2261-2268 (1997); Greelish et al., Nature Med, 5:439-443 (1999); Wang et al., Proc. Natl. Acad. Sci. USA, 96:3906-3910 (1999); Snyder et al., Nature Med., 5:64-70 (1999); Herzog et al., Nature Med, 5:56-63 (1999)); retrovirus vectors (Donahue et al., Nature Med., 4:181-186 (1998); Shackleford et al., Proc. Natl. Acad. Sci. USA, 85:9655-9659 (1988); U.S. Pat. Nos. 4,405,712, 4,650,764 and 5,252,479, and WIPO publications WO 92/07573, WO 90/06997, WO 89/05345, WO 92/05266 and WO 92/14829; and lentivirus vectors (Kafri et al., Nature Genetics, 17:314-317 (1997)). Other vectors and methods of use for introducing and expressing heterologous nucleic acids are well known in the art and can similarly be employed for the production of variant polypeptides encoded by modified parental nucleic acids of the invention.

It is understood that modifications which do not substantially affect the activity of the various embodiments of this invention are also included within the definition of the invention provided herein. Accordingly, the following examples are intended to illustrate but not limit the present invention.

### EXAMPLE I

### Combinatorial Mutagenesis and Screening of Tobacco 5-Epi-Aristolochene Synthase

This Example describes combinatorial mutagenesis for the synthesis of a predetermined variant gene library of the terpene cyclase enzyme known as tobacco 5-epi-aristolochene synthase (TEAS).

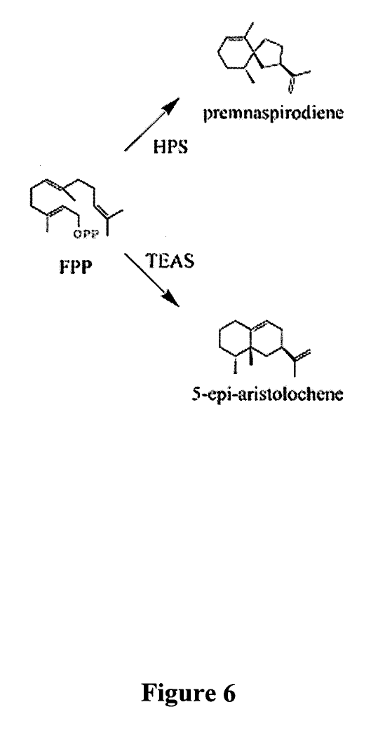

The product specificity of TEAS can be converted from 5-epi-aristolochene to premnaspirodiene through the incorporation of nine amino acid changes. Conversion of product specificity was accomplished by the sequential introduction the nine site-directed mutations designed using the three-dimensional structure of TEAS and homology modeling of HPS. Permnaspirdiene is the product of a closely related terpene cyclase from Hyoscyamus muticus known as premnaspirodiene synthase (HPS). The products TEAS and HPS cyclases are shown in FIG. 6.

SCOPE-based combinatorial mutagenesis was employed to generate a population of variant TEAS polypeptides containing all possible combinations of the nine mutations. The product specificity and kinetic properties of the variants were then analyzed to determine which mutations and what combinations of the nine mutations were sufficient to confer a change in product specificity from 5-epi-aristolochene to premnaspirodiene. The mechanistic and energetic landscape that links such a switch in product specificity to the altered amino acid residues was also assessed.

The terpene cyclases exhibit a number of attributes that can be used in either the design of variants predicted to have altered functions or the identification of structure and function relationships. For example, terpene cyclases exhibit a catalytic mechanism which employs a conformationally directed production of reactive carbocation intermediates. Terpene cyclases also exhibit well-defined three dimensional structures and generate products that are easily identified and quantified using, for example, high throughput GC-MS analysis. Further, terpene cyclases exhibit an evolutionarily diverse distribution of protein sequences and small molecule products across multiple kingdoms. In addition, the generation of functionally altered terpene cyclases have practical advantages in the biosynthesis of unique repertoires small molecules that can be useful in the diagnosis and treatment of a variety of diseases.

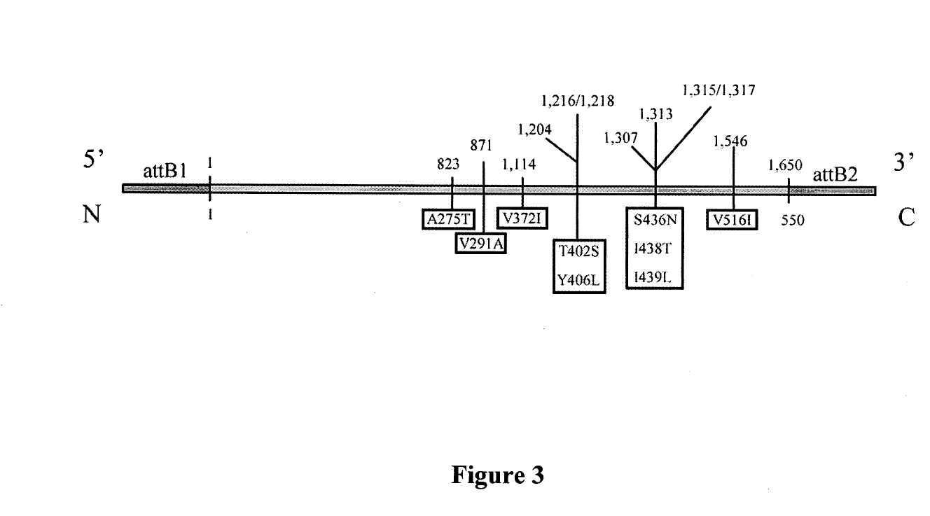

Constructing a population of all combinations of mutant sequences yields 2ⁿ different variants, where n corresponds to the number of mutations. For the nine TEAS mutations the variant population corresponds to 2⁹ or 512 different TEAS variant sequences. The location of mutations in the amino acid and nucleotide sequences of TEAS are indicated in FIG. 3. The nine amino positions were recombined as six units indicated in the boxes of FIG. 3. Some mutations were clustered, requiring a plurality of internal primers. For example, amino acid positions 436, 438, and 439 required a collection of seven internal primers to code for all permutations; 3 single, 3 double, and a triple mutant.

A nomenclature and hierarchical organizational system was developed to introduce unique sequence tags and identify specific variants in the resultant product population. PAPs containing a unique sequence tags were used to link a mutation, or a collection of mutations, to the associated tag during gene fragment amplification. SAPs were employed to selectively amplify any of designed combinations of mutations. An illustration of the nomenclature, organizational system and their use in identifying a particular variant is shown in FIG. 4.

One attribute of the combinatorial mutagenesis methods of the invention is the efficient fractionation of complex mixtures into many simpler ones. This attribute has the benefit of reducing the numerical complexity, and hence, the screening requirements necessary to verify and identify the collection of desired changes. This benefit arises from sampling probability as described by the following mathematical expression:

$$p ( n ) = 1 - {\sum }_{i = 1}^{n - 1} {( - 1 )}^{i + 1} {\frac{n !}{i ! ( n - i ) !} [ \frac{( n - i )}{n} ]}^{k} \qquad ( 1 )$$

where k is the sample size, n is the number of unique members, and p is the probability that a sample of size k contains at least one representative of each unique member.

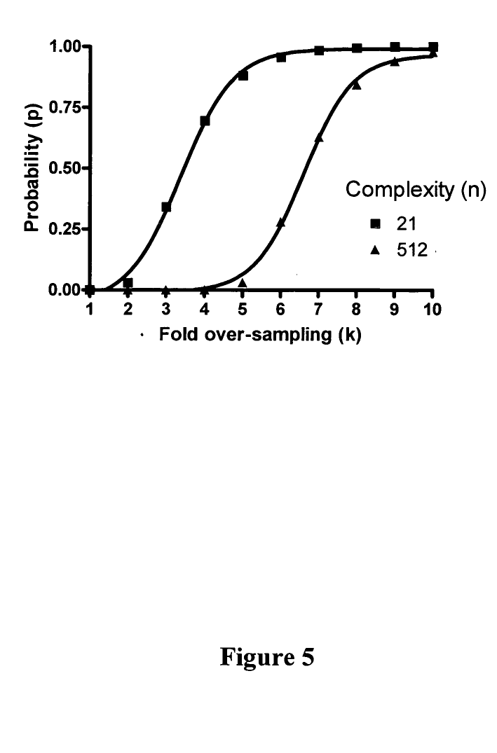

As complexity increases, the amount of over-sampling required to achieve the same probability of screening the library increases. Over-sampling refers to sample size (k) in multiples of library complexity (n). This correlation between complexity and over-sampling is shown graphically in FIG. 5. For the TEAS variant population described in this example, 512 mutants were made from a series of simpler mixtures. The most complex mixture of such simpler subsets contained 21 unique members. Employing equation (1), to achieve a 50% probability (p=0.5) of identifying by screening every unique member in a mixture having a complexity of 21 requires 3.38-fold over-sampling. To achieve the same probability of identifying all unique members of a library by screening a mixture of 512 unique possibilities requires 6.6-fold over-sampling. Given the exponential relationship between sample size and library complexity, this difference equates to a reduction in numerical complexity of a factor of 25 for the entire library.

PCR reactions for combinatorial mutagenesis were carried out using a master mix of standard set of PCR components for a 50 μl scale reaction. PCR components consisted of: 10× cloned pfu reaction buffer and pfu turbo DNA polymerase (Stratagene, la Jolla, Calif.), dNTPs (Invitrogen, Carlsbad, Calif.), and BSA (New England Biolabs, Beverly Mass.). PCR reactions were carried out using a PTC 200 Peltier Thermal Cycler (MJ Research, Waltham, Mass.). All PCR products were purified by gel extraction (Qiagen, Valencia, Calif.), cloned into pDONRTM207 using Gateway cloning technology™ (Invitrogen, Carlsbad, Calif.) according to manufacturer recommended conditions. Plamid DNA from gentamicin resistant transformants was minipreped by the Salk Institute Microarray facility for sequencing at the Salk Institute DNA Sequencing/Quantitative PCR Facility. The cDNA of TEAS was cloned into pH8GW (an in-house gateway destination vector) and this plasmid DNA was used as template for PCR.

A 50 μl scale reaction consisted of the following mixtures of PCR components. Five μl of 10× cloned pfu reaction buffer to give 1×. One μl of pfu turbo DNA polymerase (Stratagene, la Jolla, Calif.) (2.5 U/μl) to give 0.05 U/μl and 0.5 μl of BSA (10 mg/ml) to give 0.1 mg/ml. The reaction also contained 8 μl of dNTP mix (1.25 mM) to give 200 μM each dNTP.

Oligonucleotide primers used in the PCR reactions were purchased from Integrated DNA Technologies (IDT) and are listed below in Table I. For both mutagenic and chimeric primers, the mutation(s) or crossover point(s) are located in the center of the oligonucleotide, such that flanking sequence is complementary to a given parental or target gene. Generally, the oligonucleotide primers were between about 18 to 24 nucleotides and had a Tm greater than or equal to 50° C., which resulted in efficient priming and PCR amplification. SAPs were designed to consist of about 21 nucleotides and have a Tm greater than or equal to 55° C. PAPs contained about 24 bases additional to their unique sequence tag, which corresponded to Gateway™ attB sites. Tm values were calculated based on nearest-neighbor thermodynamic parameters.

Gel electrophoresis was used for analysis of PCR fragments. Separation of products for gel purification was performed using 2% (w/v) agarose gels in 1× TAE buffer containing 0.1 μg/ml ethidium bromide. Concentrations of PCR products (obtained in step IB and step III) were estimated by comparison to a standard of known concentration such as the low DNA mass ladder (Invitrogen, Carlsbad, Calif.) using densitometry software such as ImageJ (found at the url://rsb.info.nih.gov/ij/).

Prior to library construction, all primers were tested to ensure they result in unique amplification products of the expected size. Some PCR amplification reactions were optimized using well known procedures such as adjusting cycling parameters or primer sets employed for a particular template. Specific parameters for each step of the SCOPE-based combinatorial mutagenesis is described below.

[image]

[image]

[image]

Step IA consists of synthesis of the mutagenic/chimeric ssDNA incorporating the desired mutations into the amplification product. This synthesis is exemplified in FIG. 2B. Briefly, reactions were mixed on ice and included the addition of 14.5 μl of PCR master mix; 1 μl internal primer (5 μM stock) to give 0.1 μM; 1 μl plasmid DNA template (10 nM stock) to give approximately 200 pM; 33.5 μl filter-sterilized H₂O added to give 50 μl reaction volume. Master mix was added last and the resultant reaction was mixed by pipetting. Cycling parameters for amplification consisted of: 96° C. for five minutes, followed by 50 cycles of 96° C. for 30 seconds, 55° C. for 30 seconds, and 72° C. for one minute/Kb of product followed by incubation at 4° C. at the completion of cycling.

Analysis of the step IA reaction products showed that the amount of single-stranded product formed was limited by the amount of template DNA and the number of cycles performed. Estimated yields for the above reaction (using 50 cycles and approximately 200 pM plasmid) were about 10 fmols of final single stranded product. This amount is well in excess of what is required for subsequent amplification reactions. A 0.1 μM concentration of internal primer (>10³ molar excess of plasmid template) is sufficient. Higher primer concentrations result in alternative product formation in the subsequent amplification steps (step IB).

A Dpn I digestion of plasmid DNA was incorporated following single stranded DNA synthesis of the mutagenic/chimeric DNA. The Dpn I reaction consisted of the addition of 1 μl of Dpn 1 (20 U/μl, New England Biolabs, Beverly Mass.) with mixing, followed by incubation at 37° C. for 1 hour for digestion of the original DNA template and 20 minutes at 80° C. for heat inactivation of the Dpn I restriction enzyme.

Following restriction digestion, Step IB is performed to synthesize the second strand of the mutagenic/chimeric molecule and amplify the product. Use of a crossover primer in this substep, allows incorporation of a heterologous sequences into the product to form the actual chimeric molecule. This synthesis and amplification is shown in FIG. 2B.

The double strand and amplification reactions were mixed on is and included the addition of 14.5 μl of PCR master mix; 2 μl internal primer (5 μM stock) to give 0.2 μM; 1 μl primary amplification primer (5 μM stock) to give 0.1 μM; 1 μl of step IA reaction as template to give approximately 1-10 pM single-stranded DNA, and 31.5 μl filter-sterilized H2O added to give 50 μl reaction volume. Master mix was added last with pipetting to mix reactions. Cycling parameters for amplification consisted of: 96° C. for five minutes, followed by 40 cycles of 96° C. for 30 seconds, 55° C. for 30 seconds, and 72° C. for one minute/Kb of product followed by incubation at 4° C. at the completion of cycling. Amplification products were verified by agarose gel electrophoresis.

A comparison also was performed with the Dpn I digestion in step IA omitted. In this regard, the step IB reaction was performed using the undigested step IA product as template. Since plasmid DNA is carried over into the step IB reaction, PAPs could be extended to produce wild-type single-stranded DNA as previously described and shown in FIG. 2A. As a result, wild-type genes could be efficiently amplified using a 1 μl portion of step IB as template and a PAP and SAP primer pair. If the step IB reaction was performed using a 10-fold molar excess of mutagenic primer the amount of amplifiable wild-type gene decreased markedly. Moreover, the combination of increasing the number of cycles in step IA to 100, resulting in 2-fold more template, and using a 10-fold excess of internal mutagenic primer in step IB enabled the suppression of wild-type background and a mutagenesis efficiency of 80%, as apparent from terpene cyclase libraries produced in this manner.

Further, the selectivity of amplification or the suppression of wild-type sequences also was evaluated using the step IB reaction as template. When Dpn I digestion was complete, no amplifiable wild-type product was observed. In the case where restriction digestion is omitted, wild-type product is observed.

Internal primers containing the mutations were used in excess of external primers. Keeping the concentration of external primers below saturation and increasing the number of cycles ensures their depletion. Depleting the external primers in this step provides an efficient means to suppress accumulation of wild-type sequence background arising from “long” products generated during subsequent amplification steps from the carry over of external primer. Further, step IA product could be diluted up to 10,000-fold while still providing enough template for robust amplification.

Step II of the SCOPE-based combinatorial mutagenesis consists of producing the single mutant/crossover or multiple mutant/crossover recombinants by priming a parental or intermediate sequence with a step I product and polymerase extension.

Single mutant/crossovers reactions were mixed on ice and included the addition of: 5.8 μl of PCR master mix; 1 μl of step IB reaction to give approximately 10 nM (or 1-5 ng/μl ) gene fragment; 1 μl plasmid DNA template (10 nM stock) to give ˜200 pM final (1 ng/μl for a 7 Kb plasmid), and 12.2 μl filter-sterilized H₂O added to give 20 μl reaction volume. Master mix was added last with pipetting to mix reactions. Cycling parameters for amplification consisted of: 96° C. for five minutes, followed by 15 cycles of 96° C. for 30 seconds (+2″/cycle), 55° C. for 30 seconds, and 72° C. for one minute/Kb of product followed by incubated at 4° C. at the completion of cycling.

Multiple mutant/crossovers reactions included the same components as did the single mutant/crossover reactions except that gel purified full-length mutant/chimeric gene (step III product) at approximately 1.0 ng/μl (approximately 1 nM final concentration) was substituted for plasmid DNA.

Multiplex recombination consists of simultaneous recombination reactions using appropriately designed primers in the same reaction mix. The specificity of the primer to the target sequence allows for hybridization of a plurality of primers to parent or intermediate sequences and PCR amplification. Either single or multiple mutant/crossover recombination reactions can be performed in a multiplex format. The reaction mixture included a mixture of gene fragments consisting of step IB products and corresponded to a collection of mutations or alternative crossovers. The fragments were pooled, and 1 μl was added, to give approximately 10 nM final concentration, to prime either a plasmid (parental sequence) or full-length mutant/chimeric gene (step III product) template for a subsequent recombination polymerase extension reaction.

The amount of full-length single-stranded recombination product produced in step II was found to be limited by the amount of gene fragment from step IB added to the reaction mixture. Optimal results were obtained when gene fragments were about 1- to 10-fold molar excess of the plasmid or mutant gene that it is recombining with (by primer extension reaction). Maintaining a molar excess of such gene fragment primers was particularly beneficial in instances where single mutants/crossovers were being primed because there is only one terminus that can be exploited in the following step for selective amplification. Further, optimal results also were obtained when plasmid concentration were kept to a minimum. About 10 pM was found to be a lower limit for plasmid concentration which still resulted in useful levels of amplifiable recombination product in step III.

Step III of SCOPE-based combinatorial mutagenesis consists of the selective amplification of recombination products derived in step II. The amplification was performed by PCR using external primers selective for the respective 5′ and 3′ termini of the mutantion-containing crossover products.

Amplification of single mutants/crossovers was performed similarly to the PCR amplifications or the primer extension reactions described previously for steps I or II, respectively. Briefly, reactions were mixed on ice and included the addition of: 14.5 μl of PCR master mix; 2 μl secondary amplification primer (5 μM stock) to give 0.2 μM; 2 μl primary amplification primer (5 μM stock) to give 0.2 μM; 1 μl of step II reaction as template to give approximately 100-200 pM single-stranded DNA, and 30.5 μl filter-sterilized H₂O added to give 50 μl reaction volume. Master mix was added last with pipetting to mix reactions. Cycling parameters for amplification consisted of: 96° C. for five minutes, followed by 30 cycles of 96° C. for 30 seconds, 55° C. for 30 seconds, and 72° C. for one minute/Kb of product followed by an additional 10 minutes at 72° C. and incubation at 4° C. at the completion of cycling. The amplification products were verified by agarose gel electrophoresis.

Amplification of multiple mutants/crossovers included the same components as did the single mutant/crossover reactions except that only secondary amplification primers were used.

The final step in a cycle of SCOPE-based combinatorial mutagenesis is an amplification of full-length mutant/chimeric genes with unique sequence tags at both 5′ and 3′ ends. PCR was used as the amplification method performed in this example. In the synthesis of the first generation of mutants, only one SAP was used for selective amplification. This SAP corresponded to the unique sequence of the PAP used in step IB. A PAP was directed at the opposite terminus, where it incorporated unique sequence at this terminus. Since this primer was directed to the flanking sequence of the gene, it could efficiently prime any carry-over long product (single-stranded wild-type DNA) from step IB or any plasmid from step II. Greater specificity and product yield can be achieved when the long product from step IB is eliminated and the amount of plasmid in step II is minimized because single-stranded product generated at this step has the potential to carry over into subsequent rounds of synthesis. In the step III amplification of multiple mutants/crossovers, SAP combinations were chosen to allow selective amplification of desired recombination products. Alternatively, if two gene fragments from step I derived from opposite termini of the gene are recombined in a step II reaction, then the corresponding set of SAPs can be used for selective amplification in step III.

Final chimeric mutant products were isolated and cloned into vectors to produce a library of TEAS variants. Briefly, full-length mutant genes from step III were gel-purified using the Qiagen gel extraction kit according to manufacturer recommended procedures. Gel-purified attB PCR products were cloned into pDONR207 via the gateway BP reaction according to manufacturer recommendations.

Analysis of the library of TEAS variants was performed on over 600 colonies from discrete mixtures. The more than 600 colonies represented about half of the complexity of the TEAS variant population, or 241 unique members. The colonies were picked and their nucleotide and deduced amino acid sequences determined. A summary of the results is listed in Table II.

TABLE II
Sequence analysis results.
Library statistics

| | |
|---|---|
| Clones sequenced | 692 |
| Wild-type genes | 24 |
| % of mutants | 96.5% |
| Complexity screened | 241 |
| Unique clones identified | 193 |
| Fold oversampled | 2.8 |
| Complexity covered | 80.1% |
| Total library complexity | 512 |
| % of verified mutants | 37.70% |
| additional mutations: | |
| silent | 9 |
| frame-shift | 16 |
| point mutants | 13 |
| total | 38 |
| mutation rate | 5.49% |

Of the clones sequenced, only 24 wild-type genes (3.5%) were found. This library was synthesized prior to addition of the Dpn I restriction step as described previously. While the efficiency of the first round of mutagenesis was about 80%, the overall efficiency of the entire process reached 96.5% (Table II). Mutations became incorporated into wild-type sequence during recombination reactions in subsequent iterations of the process. As a result, wild-type sequences diminished in multiple crossover populations.

Aside from the low-level appearance of wild-type sequence and random mutations likely arising from PCR errors, the actual distribution of mutations obtained in a given mixture was as experimentally designed. Some recombination reactions produced a single product having several designed mutations such as A1236. In reactions containing multiple mutations, the reaction distribution appeared random.

Each iteration of the process ends with a PCR amplification step of the entire region contained the mutations incorporated by design. However, multiple iterations resulted in the accumulation of a small percentage of unspecified mutations. The overall frequency of such undesired additional mutations in the population analyzed was 5.5%. No strong bias for the type of error or its location within the gene was observed. The undesired mutation rate after the first round was 2.67%, which matches previous measures of pfu error frequency. However, the random mutation rate increases as a function of SCOPE iterations, and after four iterations reached 8.9%. Using a higher fidelity polymerase can minimize such random mutation rates. Alternatively, products from step III amplification reactions can be isolated and the SCOPE combinatorial mutagenesis cycle started anew (from step IA). Bridging oligonucleotides also can be useful to recombine various mutations and gene fragments (from step IB) can be made to include multiple mutations from previous cycles in order to lower the undesirable mutation frequency.

The development of SCOPE-based combinatorial mutagenesis for design and construction of diverse populations of specified variant nucleotide and encoded amino acid sequences demonstrates the flexibility of this method for use in a broad range of different applications. While previous methods have been developed for either homology-independent recombination or, alternatively, combinatorial mutagenesis, none have been able to efficiently do both. In contrast, SCOPE-based combinatorial mutagenesis provides an effective means for both the creation of global or local sequence variants.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

1. A method for the combinatorial mutagenesis of a parental nucleic acid, comprising:
(a) extending by enzymatic polymerization a first mutagenic primer annealed to a parental nucleic acid to produce an extension product;
(b) treating said extension product with a cleaving reagent selective for a nucleotide sequence present in the parental nucleic acid but absent in the first product;
(c) extending by enzymatic polymerization a first PAP annealed to a noncontiguous region of said mutagenic primer to produce a first product having a first mutagenized portion comprising one or more altered nucleotides, the first PAP containing a unique sequence tag associating mutations within the first mutagenic primer with the first PAP
(d) annealing the first product to the parental nucleic acid, and
(e) extending by enzymatic polymerization the annealed first product to produce a first modified parental nucleic acid containing a first mutagenized portion.

2. The method of claim 1, further comprising the step:
(c1) amplifying the first product.

3. The method of claim 1, further comprising the step:
(f) amplifying the first modified parental nucleic acid containing a first mutagenized portion by polymerase extension of an annealed first SAP to the unique sequence tag contained in the first PAP and an annealed second PAP to the first modified parental nucleic acid, the first and second PAPs corresponding to flanking regions of the parental nucleic acid.

4. The method of claim 3, further comprising the steps:
(g) repeating steps (a) through (c) one or more times with a second mutagenic primer and a third PAP to noncontiguous regions of the parental nucleic acid to a second product having a second mutagenized portion, the third PAP containing a unique sequence tag associating mutations within the second mutagenic primer with the second PAP, and
(h) repeating steps (d) through (e) or steps (d) through (f) one or more times by annealing the second product produced in step (g) to the parental nucleic acid or the first modified parental nucleic acid produced in step (e) or (f) to generate a second modified parental nucleic acid containing a first mutagenized portion and at least one second mutagenized portion.

5. The method of claim 4, further comprising the step:
(h) repeating steps (g) and (h) at least once with one or more tertiary mutagenic primers and tertiary PAPs to generate a tertiary modified parental nucleic acid containing first, second and tertiary mutagenized portions.

6. The method of claim 1, wherein the first or second mutagenized portions comprise one or more mutations.

7. The method of claim 1, wherein the first or second mutagenized portions comprise two or more mutations.

8. The method of claim 1, wherein the first and second mutagenized portions comprise two or more mutations.

9. The method of claim 1, wherein the second modified parental nucleic acid encodes between about 3-25 amino acid changes.

10. The method of claim 1, wherein the second modified parental nucleic acid encodes between about 4-20 amino acid changes.

11. The method of claim 5, wherein the one or more tertiary mutagenized portions comprise two or more mutations.

12. The method of claim 5, wherein the tertiary modified parental nucleic acid encodes between about 3-10⁴ amino acid changes.

13. The method of claim 5, wherein the tertiary modified parental nucleic acid encodes between about 26-10³ amino acid changes.

14. The method of claim 5, wherein the tertiary modified parental nucleic acid encodes greater than about 500 amino acid changes.

15. The method of claim 5, wherein the tertiary modified parental nucleic acid encodes greater than about 10⁴ amino acid changes.

16. The method of claim 1 wherein the mutagenic primers comprise random or degenerate nucleotide sequences.

17. The method of claim 1, wherein the mutagenic primers encode random, biased or predetermined amino acid sequences.

18. The method of claim 1, wherein a mutagenic primer comprises a bridging oligonucleotide.

19. The method of claim 1, wherein the parental nucleic acid comprises a single nucleic acid species.

20. The method of claim 1, wherein the parental nucleic acid comprises two or more different nucleic acid species.

21. The method of claim 20, further comprising annealing a chimeric oligonucleotide in step (a).

22. The method of claim 1, 4 or 5, wherein the first, second or tertiary modified parental nucleic acid comprises a parental nucleic acid.

23. A method for the combinatorial mutagenesis of a parental nucleic acid, comprising:
(a) extending by enzymatic polymerization a plurality of first mutagenic primers annealed to a parental nucleic acid to produce a plurality of extension products;
(b) treating the plurality of extension products with a cleaving reagent selective for a nucleotide sequence present in the parental nucleic acid but absent in the plurality of first products;
(c) extending by enzymatic polymerization a first PAP a plurality of first PAPs annealed to noncontiguous regions from said mutagenic primers to produce a plurality of first products each having a first mutagenized portion comprising one or more altered nucleotides, each of the plurality of first PAPs containing a unique sequence tag associating mutations within each of the first mutagenic primers with the plurality of first PAPs;
(d) annealing the plurality of first products to the parental nucleic acid, and
(e) extending by enzymatic polymerization the annealed plurality of first products to produce a plurality of first modified parental nucleic acids containing a first mutagenized portion.

24. The method of claim 23, further comprising the step:
(c1) amplifying the plurality of first products.

25. The method of claim 23, further comprising the step:
(f) amplifying the plurality of first modified parental nucleic acids containing a first mutagenized portion by polymerase extension of an annealed plurality of first SAPs to the unique sequence tag contained in the plurality of first PAPs and an annealed plurality of second PAPs to the first modified parental nucleic acid, the plurality of first and second PAPs corresponding to flanking regions of the parental nucleic acid.

26. The method of claim 25, further comprising the steps:
(g) repeating steps (d) through (e) or steps (d) through (f) one or more times by annealing the plurality of first products produced in step (c) to the plurality of first modified parental nucleic acids produced in step (e) to generate a plurality of second modified parental nucleic acids containing a first mutagenized portion and at least one second mutagenized portion.

27. The method of claim 23, further comprising the step:
(h) repeating step (g) at least once by annealing the plurality of first products to the plurality of first or second modified parental nucleic acids and a plurality of tertiary PAPs to generate a plurality of tertirary modified parental nucleic acids containing first, second and tertiary mutagenized portions.

28. The method of claim 23, wherein the first or second mutagenized portions comprise one or more mutations.

29. The method of claim 23, wherein the first or second mutagenized portions comprise two or more mutations.

30. The method of claim 23, wherein the first and second mutagenized portions comprise two or more mutations.

31. The method of claim 23, wherein the plurality of second modified parental nucleic acids each encode between about 3-25 amino acid changes.

32. The method of claim 23, wherein the plurality of second modified parental nucleic acids each encode between about 4-20 amino acid changes.

33. The method of claim 27, wherein the one or more tertiary mutagenized portions comprise two or more mutations.

34. The method of claim 27, wherein the plurality of tertiary modified parental nucleic acids each encode between about 3-10⁴ amino acid changes.

35. The method of claim 27, wherein the plurality of tertiary modified parental nucleic acids each encode between about 26-10³ amino acid changes.

36. The method of claim 27, wherein the plurality of tertiary modified parental nucleic acids each encode greater than about 500 amino acid changes.

37. The method of claim 27, wherein the plurality of tertiary modified parental nucleic acids each encode greater than about 10⁴ amino acid changes.

38. The method of claim 23 wherein the mutagenic primers comprise random or degenerate nucleotide sequences.

39. The method of claim 23, wherein the mutagenic primers encode random, biased or predetermined amino acid sequences.

40. The method of claim 23, wherein a mutagenic primer comprises a bridging oligonucleotide.

41. The method of claim 23, wherein the parental nucleic acid comprises a single nucleic acid species.

42. The method of claim 23, wherein the parental nucleic acid comprises two or more different nucleic acid species.

43. The method of claim 42, further comprising annealing a chimeric oligonucleotide in step (a).

44. The method of claim 23, 26 or 27, wherein the first, second or tertiary modified parental nucleic acid comprises a parental nucleic acid.

45. A hierarchical classification system associating sequences between a mutagenic and a noncontiguous parental region of a nucleic acid, comprising:
(a) a recombination matrix indexing a plurality of 5′ and 3′ unique sequence tags associated with a plurality of mutagenic primer sequences,
the indexing relating a 5′ unique sequence tag, one or more mutagenic sequences and a 3′ unique sequence tag,
wherein a 5′ or a 3′ unique sequence tag identifies a mutagenic sequence incorporated into a parental nucleic acid sequence, and wherein both 5′ and 3′ unique sequence tags identify a combination of mutagenic sequences incorporated into a parental nucleic acid.

46. The hierarchical classification system of claim 45, wherein the 5′ and 3′ unique sequence tags are indexed to a single mutagenic sequence.

47. The hierarchical classification system of claim 45, wherein the 5′ and 3′ unique sequence tags are indexed to two mutagenic sequences.

48. The hierarchical classification system of claim 45, wherein the 5′ and 3′ unique sequence tags are indexed to three or more mutagenic sequences.

49. A method of deconvoluting a plurality of mutations introduced into a parental nucleic acid sequence, comprising:
(a) forming a recombination matrix indexing a plurality of 5′ and 3′ unique sequence tags to a mutagenic primer sequence;
(b) amplifying a plurality of modified parental nucleic acid sequences having a plurality of incorporated mutations associated with one or more unique sequence tags corresponding to 5′, 3′ or both 5′ and 3′ noncontiguous regions compared to a region of complementarity to the mutagenic primer, the amplification using a pair of SAPs corresponding to the unique sequence tags, and
(c) correlating the amplification products obtained with each SAP of the pair of SAPs to its associated mutagenic primer sequence to identify the plurality of incorporated mutations within a modified parental nucleic acid sequence.

50. The method of claim 49, wherein the 5′ and 3′ unique sequence tags are indexed to a single mutagenic primer sequence.

51. The method of claim 49, wherein the 5′ and 3′ unique sequence tags are indexed to two mutagenic primer sequences.

52. The method of claim 49, wherein the 5′ and 3′ unique sequence tags are indexed to three or more mutagenic primer sequences.
